(19) United States
(12) Patent Application Publication
(10) Pub. No.: US 2020/0165312 A1
(43) Pub. Date: May 28, 2020

(54) COSMETIC COMPOSITION FOR IMPROVING SKIN CONDITIONS COMPRISING FUSION PROTEIN INCLUDING SKIN PENETRATION ENHANCING PEPTIDE

(71) Applicant: LG HOUSEHOLD & HEALTH CARE LTD., Seoul (KR)

(72) Inventors: Jin Hyun KIM, Daejeon (KR); Jaeyoon KIM, Daejeon (KR); Ho Song CHO, Daejeon (KR); Ji Seon YOON, Daejeon (KR); Sanghwa LEE, Daejeon (KR); Nae Gyu KANG, Daejeon (KR); Hyun Jong LEE, Daejeon (KR); Seol Hoon LEE, Daejeon (KR); Eugene HUR, Daejeon (KR); Taeyoon KIM, Daejeon (KR)

(73) Assignee: LG HOUSEHOLD & HEALTH CARE LTD., Seoul (KR)

(21) Appl. No.: 16/627,116

(22) PCT Filed: Jun. 28, 2018
(86) PCT No.: PCT/KR2018/007370

§ 371 Date: Dec. 27, 2019

(30) Foreign Application Priority Data
Jun. 28, 2017 (KR) 10-2017-0081712
Dec. 18, 2017 (KR) 10-2017-0174500
Dec. 18, 2017 (KR) 10-2017-0174501
Dec. 18, 2017 (KR) 10-2017-0174502
Dec. 18, 2017 (KR) 10-2017-0174616
Dec. 18, 2017 (KR) 10-2017-0174617

(51) Int. Cl. C07K 14/485; C07K 14/575; A61K 8/64; A61P 17/14; A61Q 7/00; C07K 14/49; C07K 14/65; A61Q 19/08; A61K 38/00

(52) U.S. Cl. CPC C07K 14/485; C07K 14/57581; A61K 8/64; A61P 17/14; A61K 38/00; C07K 14/49; C07K 14/65; A61Q 19/08; A61Q 7/00

(57) ABSTRACT

Provided is a cosmetic composition for improving skin conditions including a fusion protein containing a skin penetration enhancing peptide, more particularly, a fusion protein in which PDGFa is bound to the skin penetration enhancing peptide, and a cosmetic composition for improving skin conditions, a functional cosmetic product for improving skin conditions, a cosmetic composition for preventing and treating alopecia, and a quasi-drug composition each including the fusion protein.

### TECHNICAL FIELD

The present invention relates to a cosmetic composition for improving skin conditions including a fusion protein including a skin penetration enhancing peptide, more particularly, to a fusion protein in which a physiologically active protein is bound to a skin penetration enhancing peptide, and a cosmetic composition for improving skin conditions, a functional cosmetic product for improving skin conditions, a cosmetic composition for preventing and treating alopecia, and a quasi-drug composition each including the fusion protein.

### BACKGROUND ART

Drugs penetrating skin have been used in analgesic patches, nicotine patches, birth control patches, and the like, because of convenience in use. Drug delivery through skin mainly involves the delivery through the skin into systemic circulation. Also, drugs such as therapeutic agents for atopic dermatitis, cosmetics for whitening or anti-wrinkle effects are used for the transport of drugs into the skin organ itself. Despite the convenience and functionality, there are many difficulties in drug delivery through the skin, due to the structure of skin. The stratum corneum is an epidermal layer consisting of about 10 to 15 layers of corneocytes having a thickness of about 10 μm to 45 μm and has a brick and mortar structure including a brick structure composed of keratin-rich corneocyte bricks and a mortar structure composed of lipids such as ceramide, fatty acid, or wax which fills the space between corneocytes. This structure prevents loss of internal moisture from skin surface and external attacks. Thus, this structure has the property of very low permeability as faithfully corresponding to functions thereof. Only substances having a low molecular weight below 500 Da may pass through the skin by diffusion (Exp. Dermatol., 2000, 9, 165-9.). This may be performed through the intracellular lipid layer of the mortar structure or the hydrophilic structure between lipid layers. Drug delivery through the skin may be greatly influenced by properties of drugs (Current Drug Delivery, 2005, 2, 23-3). In addition, drug delivery through the skin may also occur through other structures such as sweat glands, skin pores, sebaceous glands, in addition to the direct passage through the skin surface.

For this reason, extensive research has been carried out to develop a method transmitting and uniformly delivering a substance through the skin regardless of size or property of molecules thereof.

For example, U.S. Pat. No. 7,659,252 discloses a transdermal peptide that may be used to enhance skin penetration of a therapeutic agent and a pharmaceutically active agent for skin disease.

Although the peptide may be used as a carrier for transdermal delivery of other drugs in addition to excellent skin penetrability, the peptide is consumed through the circulation system after penetrating the skin, and thus the effect of the peptide is negligible for drugs to be delivered to the skin as a target of the delivery.

Various active ingredients exhibiting skin wrinkle reduction effects by promoting collagen synthesis, endothelial cell growth, or hyaluronic acid production cannot be absorbed through the skin. Due to these disadvantages, studies have been extensively conducted to develop methods of promoting absorption of the active ingredients. For example, Korean Patent No. 1054519 discloses a human growth hormone-derived peptide having excellent stability and skin penetrability compared to natural human growth hormones and a composition including the same, and Korean Patent No. 1104223 discloses an IL-10-derived peptide, which performs the same function as that of human IL-10 and has excellent stability and skin penetrability compared to natural IL-10 and a composition including the same. However, these peptides have a disadvantage in that they merely exhibit functionality by themselves and cannot be used as carriers for delivering other drugs. This disadvantage suggests that excellent skin penetrability alone does not satisfy the requirements for drug delivery. Thus, there is still a need to develop novel materials that satisfy both excellent skin penetrability and excellent skin retentivity, but results of studies on such materials have not been reported, yet.

### DESCRIPTION OF EMBODIMENTS

### Technical Problem

Under such backgrounds, as a result of intensive efforts to develop novel substances having excellent skin retentivity as well as excellent skin penetrability, the present inventors have found a skin penetration enhancing peptide that may be used as a carrier for transdermal delivery of drugs and remain in the skin for a long time and have identified that a fusion protein in which a physiologically active protein is bound to a skin penetration enhancing peptide has excellent skin retentivity as well as excellent skin penetrability, thereby completing the present invention.

### Solution to Problem

An object of the present invention is to provide a fusion protein including a skin penetration enhancing peptide comprising an amino acid sequence of SEQ ID NO: 1 and a physiologically active protein.

Another object of the present invention is to provide a polynucleotide encoding the fusion protein.

Another object of the present invention is to provide a cosmetic composition for improving skin conditions including the fusion protein as an active ingredient.

Another object of the present invention is to provide a functional cosmetic product for improving skin conditions including the cosmetic composition as an active ingredient.

Another object of the present invention is to provide a cosmetic composition for preventing and treating alopecia including the fusion protein as an active ingredient.

Another object of the present invention is to provide a quasi-drug composition for improving skin conditions including the fusion protein as an active ingredient.

Another object of the present invention is to provide a quasi-drug composition for preventing and treating alopecia including the fusion protein as an active ingredient.

### Advantageous Effects of Disclosure

Since a skin penetration enhancing peptide is bound to a physiologically active protein in the fusion protein according to the present invention, the fusion protein may not only maintain or improve the ability of the peptide exhibiting useful effects such as wrinkle reduction but also improve both skin penetrability and skin retentivity. Therefore, the fusion protein may be widely used as an active ingredient of cosmetic compositions for improving skin conditions, functional cosmetic products for improving skin conditions, cosmetic compositions for preventing and treating alopecia, quasi-drug compositions for improving skin conditions, or a quasi-drug composition for preventing and treating alopecia.

### BRIEF DESCRIPTION OF DRAWINGS

The above and other aspects, features, and advantages of certain embodiments of the present invention will be more apparent from the following description taken in conjunction with the accompanying drawings.

FIG. 1 shows effects of Argireline™ (Acetyl-EEMQRR SEQ ID NO: 2) and a neurotransmitter release regulating fusion protein (SEQ ID NO: 32) on inhibition of muscle contraction.

FIG. 2 is a graph illustrating effects of an Argireline™ (Acetyl-EEMQRR)-containing cream and a neurotransmitter release regulating fusion peptide-containing cream on wrinkle reduction of human bodies.

### BEST MODE

An aspect of the present invention to achieve the above objects provides a fusion protein in which a skin penetration enhancing peptide comprising an amino acid sequence of SEQ ID NO: 1 is bound to a physiologically active protein.

The amino acid sequence of SEQ ID NO: 1 used herein is abbreviated as follows according to the IUPAC-IUB nomenclature.

Skin penetration enhancing peptide: NGSLNTHLAPIL (SEQ ID NO: 1)

Specifically, the abbreviations are as follows: Asparagine (Asn, N), Glycine (Gly, G), Serine (Ser, S), Leucine (Leu, L), Asparagine (Asn, N), Threonine (Thr, T), Histidine (His, H), Leucine (Leu, L), Alanine (Ala, A), Proline (Pro, P), Isoleucine (Ile, I), and Leucine (Leu, L).

The physiologically active protein bound to the skin penetration enhancing peptide may be a neurotransmitter release regulating peptide, a platelet-derived growth factor subunit a (PDGFa), a vascular endothelial growth factor (VEGF), an insulin-like growth factor-1 (IGF-1), a keratinocyte growth factor (KGF), or a thymosin beta 4 (Tβ4) and may have skin penetrability and skin retentivity.

As used herein, the term “skin penetration enhancing peptide” refers to a peptide that penetrates skin regardless of molecular size or characteristic thereof, uniformly spreads throughout the skin, and has excellent skin penetrability and excellent skin retentivity.

In the fusion protein of the present invention, skin penetrability and skin retentivity of the physiologically active protein may be improved.

Throughout the specification, the term “skin penetrability” refers to the ability or characteristic of a peptide to penetrate and permeate the skin, and the skin penetration enhancing peptide according to the present invention has remarkably superior skin penetrability to other peptides.

As used herein, the term “skin retentivity” refers to the ability of a peptide to penetrate skin to bind to skin tissue, thereby remaining in the skin without being delivered to the circulatory system through the skin tissue. Pharmaceutical formulations or cosmetic formulations targeting skin tissue may use a peptide that has an excellent property of remaining in skin tissue as a carrier so that a component bound to the peptide may act on skin tissue or skin cells for a long time.

Since the skin penetration enhancing peptide according to the present invention has excellent skin retentivity as well as excellent skin penetrability, it may be used as a carrier for pharmaceutical formulations or cosmetic formulations.

The skin penetration enhancing peptide of the present invention may include a peptide having excellent skin penetrability and skin retentivity and excavated by performing a phage display method consisting of a combination of elution test methods using a phage library and a transdermal agent, specifically a peptide comprising an amino acid sequence of SEQ ID NO: 1. In an embodiment of the present invention, a peptide comprising the amino acid sequence of SEQ ID NO: 1 was prepared as the skin penetration enhancing peptide by a phage display method (Example 1).

As used herein, the term “physiologically active protein” refers to all proteins that are used for therapeutic effects.

Preferably, the physiologically active protein used herein collectively refers to proteins that regulate biological functions (physiological functions) and may be interchangeably used with the term physiologically active polypeptide. The physiologically active protein of the present invention may be any protein that may be used to treat the skin without limitation and any derivative of the physiologically active protein also falls within the scope of the physiologically active peptide as long as it has substantially the same or enhanced function, structure, activity, or stability compared to a wild-type physiologically active polypeptide.

More specifically, the physiologically active protein may be neurotransmitter release regulating peptide, platelet-derived growth factor subunit a (PDGFa), vascular endothelial growth factor (VEGF), insulin-like growth factor-1 (IGF-1), keratinocyte growth factor (KGF), or thymosin beta 4 (Tβ4).

As used herein, the term “neurotransmitter” refers to a series of substances released from nerve cells in a living body including a brain and transmitting information to adjacent nerve cells, i.e., an endogenous chemical substance transmitting a signal across a synapse from one neuron to another ‘target’ neuron. The neurotransmitters packed into synaptic vesicles that cluster beneath the axon terminal membrane on the presynaptic side of a synapse are released into the synaptic cleft and move across the synaptic cleft. In this case, the neurotransmitters are bound to a membrane's specific receptor in the postsynaptic side of the synapse. The neurotransmitter according to the present invention may be dopamine, serotonin, histamine, acetylcholine, adrenaline, noradrenaline, gamma-aminobutyric acid (GABA), L-glutamic acid, glycine, and the like, without being limited thereto.

In addition, as used herein, the term “neurotransmitter release regulating peptide” refers to a peptide that blocks transmission of a neurotransmitter to a receptor of the neurotransmitter, resulting in inhibition of muscle contraction, thereby reducing wrinkles. More particularly, a neurotransmitter needs to be delivered from nerve cells to muscle cells through synapses to move muscles. A process of forming a SNARE complex at the terminals of the nerve cells and releasing the SNARE complex into synapses to release acetylcholine, as the neurotransmitter. Botox, commonly known in the art, cleaves a component (SNAP-25) that forms the SNARE complex to inhibit the release of acetylcholine at synapses. However, a Botox-like peptide has a structure similar to that of SNAP-25 that is a part of the component of the SNARE complex and is involved in formation of the SNARE complex instead of the SNAP-25, thereby inhibiting the release of acetylcholine.

The neurotransmitter release regulating peptide of the present invention may be any type of peptides well known in the art without limitation. Not only natural peptides but also chemically synthesized peptides may be used. In addition, derivatives of any peptides known to have anti-wrinkle effects may also be within the scope of the present invention.

Specifically, the neurotransmitter release regulating peptide may include at least one peptide selected from the group consisting of Argireline™ (Acetyl-Glu-Glu-Met-Gln-Arg-Arg, Acetyl-EEMQRR, SEQ ID NO: 2), X50 Myocept™ manufactured by Infinitec, Palmitoyl-hexapeptide-52 ([Pal]-Asp-Asp-Met-Gln-Arg-Arg, [Pal]DDMQRR, SEQ ID NO: 3), Palmitoyl-heptapeptide-18 ([Pal]-Tyr-Pro-Trp-The-Gln-Arg-Phe, [Pal]YPWTQRF, SEQ ID NO: 4)), GABA (γ-amino butyric acid), botulinum toxin, or any mixture thereof, without being limited thereto.

In addition, the neurotransmitter release regulating peptide of the present invention may include a peptide represented by Formula 1 below, isomers or racemic compounds thereof, or cosmetically or pharmaceutically acceptable salts thereof.

R₁-AA-R₂ [Formula 1]

In Formula 1, AA is an amino acid sequence including 3 to 40 amino acids, R₁ is H or a C₃-C₂₄ alkyl, aryl, or acyl group.

In the peptide of Formula 1 above, R₁ may be an C₃-C₂₄ acyl group which is saturated or unsaturated and a linear, branched, or cyclic group.

Specifically, R₁ may be an acyl group represented by CH₃—(CH₂)ₘ—CO—, where m is an integer of 1 to 22, more particularly, R₁ may be a polyethylene glycol polymer having a molecular weight of 200 to 35,000 Da, but is not limited thereto.

In addition, the AA may comprise an amino acid sequence selected from the group consisting of MAEDADMRNELEEMQRRADQL (SEQ ID NO: 5), ADESLESTRRMLQLVEESKDAGI (SEQ ID NO: 6), ELEEMQRRADQLA (SEQ ID NO: 7), ELEEMQRRADQL (SEQ ID NO: 8), ELEEMQRRADQ (SEQ ID NO: 9), ELEEMQRRAD (SEQ ID NO: 10), ELEEMQRRA (SEQ ID NO: 11), ELEEMQRR (SEQ ID NO: 12), LEEMQRRADQL (SEQ ID NO: 13), LEEMQRRADQ (SEQ ID NO: 14), LEEMQRRAD (SEQ ID NO: 15), LEEMQRRA (SEQ ID NO: 16), LEEMQRR (SEQ ID NO: 17), EEMQRRADQL (SEQ ID NO: 18), EEMQRRADQ (SEQ ID NO: 19), EEMQRRAD (SEQ ID NO: 20), EEMQRRA (SEQ ID NO: 21), EEMQRR (SEQ ID NO: 22), LESTRRMLQLVEE (SEQ ID NO: 23), NKDMKEAEKNLT (SEQ ID NO: 24), KNLTDL (SEQ ID NO: 25), IMEKADSNKTRIDEANQRATKMLGSG (SEQ ID NO: 26), SNKTRIDEANQRATKMLGSG (SEQ ID NO: 27), TRIDEANQRATKMLGSG (SEQ ID NO: 28), DEANQRATKMLGSG (SEQ ID NO: 29), NQRATKMLGSG (SEQ ID NO: 30) and QRATKMLGSG (SEQ ID NO: 31). In addition, the AA may include an amino acid sequence derived from an amino group domain and a carboxyl group domain of the SNAP-25 protein.

In addition, R₂ of the peptide of Formula 1 may be a C₁-C₂₄ aliphatic or cyclic unsubstituted or substituted with at least one group selected from the group consisting of an amino group, a hydroxyl group, or a thiol group.

Specifically, as the substituents R₁ and R₂ of Formula 1 above, examples disclosed in US 2010-0021510 A1 may be used, and US 2010-0021510 A1 is incorporated herein by reference in its entity. As the compound of Formula 1, examples disclosed in US 2010-0021510 A1 may be used.

In an embodiment of the present invention, it was confirmed that the use of fusion proteins in which Acetyl-EEMQRR, Palmitoyl-DDMQRR, and Palmitoyl-YPWTQRF, as neurotransmitter release regulating peptides, are linked to the skin penetration enhancing peptide of SEQ ID NO: 1 respectively, may improve wrinkle reduction effects, compared to the neurotransmitter release regulating peptides alone, via direct inhibition of formation of the SNARE complex and indirect inhibition thereof by blocking introduction of Ca²⁺ ions into nerve cells.

As used herein, the term “platelet-derived growth factor (PDGF)” refers to a low molecular weight basic protein consisting of two peptide chain and facilitating proliferation of mesenchymal cells such as smooth muscle cells, fibroblasts, and vascular walls.

The term “platelet-derived growth factor subunit a (PDGFa)” refers to a proteinbelonging to the platelet-derived growth factor family, contained in blood platelets, and having a size of about 18 kDa. The PDGF present in platelets consists of subunit b having a size of about 14 kDa and subunit a as described above. It is known that these subunits form a homodimer PDGF-AA or PDGF-BB or a heterodimer PDGF-AB by a disulfide bond.

In the present invention, an amino acid sequence of PDGFa is not particularly limited, as long as the PDGFa promotes regeneration of damaged skin or regeneration and growth of hair by increasing production of collagen or elastin. The entire amino acid sequence of the PDGFa may be used or modified amino acid sequences or fragments thereof may also be used. Information on specific amino acid sequences of the PDGFa or nucleotide sequences of genes encoding the same are available from known database such as the NCBI GenBank. The PDGFa may be a peptide expressed as an amino acid sequence of SEQ ID NO: 35, but is not limited thereto.

As used herein, the term “vascular endothelial growth factor (VEGF)” refers to an important signaling protein involved in both vasculogenesis and angiogenesis. When blood circulation is inadequate, VEGF serves as a part of a system that restores and supplies oxygen to tissue. General functions of VEGF are to create new blood vessels during embryonic development or formation of muscles after injury and exercise, new blood vessels to bypass blocked blood vessels, or the like. However, overexpressed VEGF may cause abnormal angiogenesis.

VEGF playing an important role in angiogenesis mainly affects cells constituting vascular endothelium. In vitro, VEGF promotes mitosis and migration of vascular endothelial cells and increases microvascular permeability. VEGFs are classified into 5 types in mammals: VEGF-A, VEGF-B, VEGF-C, VEGF-D, and placenta growth factor (PIGF). The VEGF-A promotes angiogenesis, migration and mitosis of vascular endothelial cells, creation of blood vessel lumen, chemotaxis of macrophages and granulocytes, and vasodilation, and the VEGF-B promotes embryonic angiogenesis, particularly formation of myocardial tissue. The VEGF-C promotes lymphangiogenesis, and the VEGF-D is needed for the development of lymphatic vasculature surrounding lung bronchioles. The PIGF plays an important role for vasculogenesis and angiogenesis in ischemia, inflammation, wound healing, and cancer.

In the present invention, an amino acid sequence of the VEGF is not particularly limited, as long as the VEGF promotes regeneration of damaged skin or regeneration and growth of hair by inducing angiogenesis, proliferating epidermal cells, promoting migration of cells, and increasing microvascular permeability. The entire amino acid sequence of the VEGF may be used or modified amino acid sequences or fragments thereof may also be used. Information on specific amino acid sequences of the VEGF or nucleotide sequences of genes encoding the same are available from known database such as the NCBI GenBank. The VEGF may be a peptide expressed as an amino acid sequence of SEQ ID NO: 38, but is not limited thereto.

In addition, there are multiple isomers of VEGF having various lengths resulting from splicing in various positions, e.g., VEGF-189, VEGF-165, and VEGF-121. As a representative isomer, VEGF-165 has a size of about 19.2 kDa. The VEGF may be present as a homodimer by a disulfide bond or a heterodimer with PIGF that is a different growth factor protein.

As used herein, the term “insulin-like growth factor (IGF)” refers to a type of signaling protein formed of a polypeptide having a molecular weight of 7,500 and a similar structure as that of insulin. Although the insulin-like growth factor performs a similar action to that of insulin in serum, it is not inhibited by an insulin antibody, and IGF-1 and IGF-2 structures are known. Both IGF-1 and IGF-2 consist of 4 types (A to D) of polypeptide chains and have physiological actions similar to that of insulin in addition to mediating action of growth hormones in proliferation of cartilage cells or protein synthesis.

In addition, IGF-1 has a size of about 7.6 kDa and binds to an insulin-like growth factor receptor as a monomer to manipulate the mechanism in cells.

In the present invention, an amino acid sequence of the IGF-1 is not particularly limited, as long as the IGF-1 promotes regeneration of damaged skin or regeneration and growth of hair by promoting growth of keratinocytes. The entire amino acid sequence of IGF-1 may be used or modified amino acid sequences or fragments thereof may also be used.

Information on the specific amino acid sequences of the IGF-1 or nucleotide sequences of genes encoding the same are available from known database such as the NCBI GenBank. The IGF-1 may be a peptide expressed as an amino acid sequence of SEQ ID NO: 41, but is not limited thereto.

As used herein, the term “keratinocyte growth factor (KGF)”, as a signaling protein, refers to a growth factor present in the epithelialization-phase of wound healing, in which keratinocytes are covering the wound, forming the epithelium.

The KGF protein is encoded by an FGF7 gene and is a member of the fibroblast growth factor (FGF) family. FGF family members possess broad mitogenic and cell survival activities, and are involved in a variety of biological processes, including embryonic development, cell growth, morphogenesis, tissue repair, tumor growth and invasion. KGF is a potent epithelial cell-specific growth factor, whose mitogenic activity is predominantly exhibited in keratinocytes but not in fibroblasts and endothelial cells. KGF binds to a fibroblast growth factor receptor 2b (FCFR2b) to perform signaling and FGF10 is known as ‘keratinocyte growth factor 2’.

In the present invention, an amino acid sequence of KGF is not particularly limited, as long as the KGF restores skin by promoting the growth of skin cells as a key growth factor for wound healing or prevents alopecia by promoting proliferation of cells in hair follicles. The entire amino acid sequence of KGF may be used or modified amino acid sequences or fragments thereof may also be used. Information on the specific amino acid sequences of the KGF or nucleotide sequences of genes encoding the same are available from known database such as the NCBI GenBank. The KGF may be a peptide expressed as an amino acid sequence of SEQ ID NO: 44, but is not limited thereto.

As used herein, the term “thymosin beta 4 (Tβ4)” was initially isolated from the thymus gland and refers to a relatively small protein having a molecular weight of 5 kDa, composed of 43 amino acids, and found in almost all cells except for erythrocytes. Tβ4 is a protein regulating actin and binds to G-actin to inhibit polymerization of actin. Although Tβ4 is known to induce differentiation and migration of endothelial cells and angiogenesis, it has recently been reported that Tβ4 effectively acts on wound healing and is highly effective in regeneration of myocardial cells.

In the present invention, an amino acid sequence of Tβ4 is not particularly limited, as long as Tβ4 has the effects of promoting regeneration of damaged skin or regeneration and growth of hair by regulating mitosis, differentiation, and migration of cells and inducing angiogenesis, proliferating epidermal cells, promoting cell migration, or increasing microvascular permeability. The entire amino acid sequence of Tβ4 may be used or modified amino acid sequences or fragments thereof may also be used. Information on the specific amino acid sequences of the Tβ4 or nucleotide sequences of genes encoding the same are available from known database such as the NCBI GenBank. The Tβ4 may be a peptide expressed as an amino acid sequence of SEQ ID NO: 47, but is not limited thereto.

In addition, genes encoding Tβ4 are located in Chromosome Y and Chromosome X at q.21.3-q.22, and these two genes, as homologous genes, are very similar in sequence in which only 3 amino acids are different among 44 amino acids. According to an embodiment of the present invention, a fusion protein was prepared based on the genomic sequence of chromosome X. However, the embodiment is not limited to Tβ4 on the chromosome X and the fusion protein may also be prepared by using Tβ4 on the chromosome Y.

As used herein, the term “fusion protein” refers to a peptide artificially synthesized such that the skin penetration enhancing peptide is bound to another protein or peptide, specifically, a peptide including the skin penetration enhancing peptide and one selected from the group consisting of the neurotransmitter release regulating peptide, the platelet-derived growth factor subunit a (PDGFa), the vascular endothelial growth factor (VEGF), the insulin-like growth factor-1 (IGF-1), the keratinocyte growth factor (KGF), and the thymosin beta 4 (Tβ4). More specifically, the skin penetration enhancing peptide may be a peptide comprising the amino acid sequence of SEQ ID NO: 1, and the neurotransmitter release regulating peptide may include at least one peptide comprising an amino acid sequence selected from the group consisting of SEQ ID NOS: 2 to 31, specifically, may include a peptide comprising an amino acid sequence of SEQ ID NO: 2, 3, or 4, a peptide represented by Formula 1 above, an isomer or racemic compound thereof, or a cosmetically or pharmaceutically acceptable salt thereof. More specifically, the fusion peptide may comprise an amino acid sequence of SEQ ID NO: 32, 33, or 34 or a peptide including botulinum toxin+SEQ ID NO: 1. In addition, the platelet-derived growth factor subunit a (PDGFa) may comprise an amino acid sequence of SEQ ID NO: 35, the vascular endothelial growth factor (VEGF) may comprise an amino acid sequence of SEQ ID NO: 38, the insulin-like growth factor-1 (IGF-1) may comprise an amino acid sequence of SEQ ID NO: 41, the keratinocyte growth factor (KGF) may comprise an amino acid sequence of SEQ ID NO: 44, and the thymosin beta 4 (Tβ4) may comprise an amino acid sequence of SEQ ID NO: 47, without being limited thereto.

The fusion protein may include a peptide having a sequence, one or more amino acid residues of which differ from those of the wild-type amino acid sequence of each domain included therein. Amino acid exchanges that do not generally alter the specific activity thereof are known in the art. The most commonly occurring exchanges between amino acid residues are Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, and Asp/Gly. In addition, the fusion protein may include a protein having improved structural stability against heat, pH, or the like or increased activity by the mutation or modification of amino acids in the amino acid residues.

The fusion protein or proteins constituting the fusion protein may be prepared by a chemical protein synthesis method known in the art. Alternatively, it may be prepared by amplifying a gene encoding the fusion protein or the proteins by PCR or synthesizing the gene according to a known method, and then cloning the gene into an expression vector for expression.

The fusion protein of the present invention may include a linker peptide disposed between the skin penetration enhancing peptide and the physiologically active protein. Specifically, in the fusion protein, the skin penetration enhancing peptide may be linked directly or via a linker to the N-terminal of the physiologically active protein.

Specifically, the linker may be amino acids such as glycine, alanine, leucine, iso-leucine, proline, serine, threonine, asparagine, aspartic acid, cysteine, glutamine, glutamic acid, lysine, and arginine acid. Particularly, the linker may include one or more amino acids selected from among valine, leucine, aspartic acid, glycine, alanine and proline, and more particularly, 1 to 5 amino acids selected from among glycine, valine, leucine, and aspartic acid, in view of the ease of genetic engineering. For example, the fusion protein may be prepared by linking the C-terminal of the skin penetration enhancing peptide to the N-terminal of physiologically active protein by a linker consisting of amino acids (GG).

Specifically, the fusion protein of the present invention may be a peptide having an amino acid sequence selected from SEQ ID NOS: 32 to 34, 36, 39, 42, 45, and 48, but is not limited thereto.

Another aspect of the present invention provides a polynucleotide encoding the fusion protein.

The polynucleotide may include a polynucleotide encoding an amino acid sequence selected from sequences as set forth in SEQ ID NOS: 32 to 34, 36, 39, 42, 45, and 48 or a polynucleotide encoding a protein exhibiting at least 70%, specifically at least 80%, more specifically at least 90%, even more specifically at least 95%, and most specifically at least 99% homology with the sequence as long as the protein has activity similar to that of the fusion protein, but is not limited thereto. In addition, it is obvious that any polynucleotide which may be translated into the protein having an amino acid sequence selected from SEQ ID NOS: 32 to 34, 36, 39, 42, 45, and 48 or a protein having homology therewith by codon degeneracy may also be included. Alternatively, the polynucleotide may have any nucleotide sequence that is hybridized with a probe synthesized from known gene sequences, entirely or partially complementary to the nucleotide sequence under stringent conditions to encode the protein having the activity of a protein having an amino acid sequence selected from SEQ ID NOS: 32 to 34, 36, 39, 42, 45, and 48, without limitation.

Specifically, the polynucleotide according to the present invention may include one nucleotide sequence selected from SEQ ID NOS: 37, 40, 43, 46, and 49, without being limited thereto.

The term “stringent conditions” refers to conditions which permit specific hybridization between polynucleotides. Such conditions are disclosed in detail in known documents (e.g., J. Sambrook et al.). For example, the conditions may include performing hybridization between genes having a high homology, e.g., a homology of 80% or more, specifically 90% or more, more specifically 95% or more, even more specifically 97% or more, and most specifically 99% or more, without performing hybridization between genes having a homology lower than the above homologies, or performing hybridization once, specifically two or three times, under conventional washing conditions for Southern hybridization at a salt concentration and temperature of 60° C., 1×SSC, and 0.1% SDS, specifically 60° C., 0.1×SSC, 0.1% SDS, and more specifically 68° C., 0.1×SSC, and 0.1% SDS.

Hybridization requires that two polynucleotides have complementary sequences, although bases may mismatch due to stringent conditions of hybridization. The term “complementary” is used to describe the relationship between bases of nucleotides capable of hybridizing with each other. For example, with respect to DNA, adenosine is complementary to thymine, and cytosine is complementary to guanine. Thus, the present disclosure may include not only substantially similar polynucleotide sequence but also a polynucleotide fragment isolated but complementary to the entire sequence.

Particularly, the polynucleotide having homology may be detected under hybridization conditions including a hybridization process performed at 55° C. as a Tm value using the above-described conditions.

Also, the Tm value may be 60° C., 63° C., or 65° C., but is not limited thereto, and may be appropriately adjusted by those skilled in the art according to the purpose.

The degree of stringent conditions for hybridizing polynucleotides may depend on lengths of the polynucleotides and degrees of complementarity and parameters are well known in the art (Refer to Sambrook et al., supra, 9.50-9.51, 11.7-11.8).

As used herein, the term “homology” refers to a percent of sequence identity between two polynucleotide or polypeptide moieties. The homology also refers to a degree of relevance between two amino acid sequences or nucleotide sequences and may be expressed as a percentage. In the present invention, a homology sequence having identical or similar activity to the given amino acid sequence or nucleotide sequence is expressed as “% homology”. For example, homology may be identified using a standard software program which calculates parameters of score, identity and similarity, specifically BLAST 2.0, or by comparing sequences in a Southern hybridization experiment under stringent conditions as defined. Defining appropriate hybridization conditions is within the skill of the art and determined by a method known to those skilled in the art (J. Sambrook et al., Molecular Cloning, A Laboratory Manual, 2nd Edition, Cold Spring Harbor Laboratory press, Cold Spring Harbor, N.Y., 1989; F. M. Ausubel et al., Current Protocols in Molecular Biology, John Wiley & Sons, Inc., New York).

As used herein, the term “vector” refers to a DNA construct including a nucleotide sequence encoding a target polypeptide, which is operably linked to an appropriate expression regulatory sequence to express the target protein in a suitable host cell.

The regulatory sequence may include a promoter capable of initiating transcription, an optional operator sequence for regulating the transcription, a sequence encoding a suitable mRNA ribosome binding site, and a sequence regulating termination of transcription and translation. After the vector is introduced into the suitable host cell, it may replicate or function independently of the host genome, and may be integrated into the genome itself.

The vector used in the present invention is not particularly limited, as long as it is able to replicate in the host cell, and any vector known in the art may be used. Examples of conventional vectors may include a natural or recombinant plasmid, cosmid, virus and bacteriophage. For example, pWE15, M13, MBL3, MBL4, IXII, ASHII, APII, t10, t11, Charon4A, and Charon21A may be used as a phage vector or cosmid vector, and pBR type, pUC type, pBluescriptII type, pGEM type, pTZ type, pCL type, and pET type may be used as a plasmid vector. A vector available in the present invention is not particularly limited, and any known expression vectors may be used. Specifically, pDZ, pACYC177, pACYC184, pCL, pECCG117, pUC19, pBR322, pMW118, pCC1BAC, pPIC, and pGAP vectors may be used, and any vectors expressed by bacteria such as Escherichia Coli (E. Coli), lactobacillus, bacillus species and yeasts may also be used.

For example, a polynucleotide encoding a target polypeptide in the chromosome may be replaced with a modified polynucleotide via a vector inserted into the chromosome. The insertion of the polynucleotide into the chromosome may be performed by any method known in the art, for example, homologous recombination, without being limited thereto.

As used herein, the term “transformation” refers to a process of introducing a vector including a polynucleotide encoding a target polypeptide into a host cell, thereby enabling the expression of the polypeptide encoded by the polynucleotide in the host cell. The transformed polynucleotide may be either in a for inserted into the chromosome of the host cell or in a form located outside the chromosome, as long as the polynucleotide is expressed in the host cell. For example, methods for transformation may include electroporation, calcium phosphate (CaPO₄) precipitation, calcium chloride (CaCl₂) precipitation, microinjection, polyethylene glycol (PEG) method, DEAE-dextran method, cationic liposome method, and lithium acetate-DMSO method, without being limited thereto. In addition, the polynucleotide includes DNA and RNA encoding the target polypeptide. The polynucleotide may be introduced in any form, as long as it is able to be introduced into the host cell and expressed therein. For example, the polynucleotide may be introduced into the host cell in the form of an expression cassette, which is a gene construct including all elements required for autonomous expression thereof. Typically, the expression cassette includes a promoter operably linked to the polynucleotide, a transcriptional termination signal, a ribosome binding site, or a translation termination signal. The expression cassette may be in the form of a self-replicable expression vector.

Also, the polynucleotide as it is may be introduced into the host cell and operably linked to a sequence required for expression in the host cell, without being limited thereto.

As used herein, the term “operably linked” means a functional linkage between a polynucleotide sequence encoding the polypeptide of the present disclosure and a promoter sequence which initiates and mediates transcription of the polynucleotide sequence.

Another aspect of the present invention provides a cosmetic composition for improving skin conditions including the fusion protein as an active ingredient.

Specifically, a cosmetic composition for skin wrinkle reduction or skin elasticity enhancement including the fusion protein according to the present invention as an active ingredient may be provided.

As used herein, the terms “skin elasticity enhancement” or “skin wrinkle reduction” refers to decreasing the degree of skin sagginess, inhibiting or suppressing wrinkle formation, or reducing wrinkles already formed. As the amount of collagen or hyaluronic acid distributed in intercellular spaces and connective tissue of dermis increases, skin elasticity may be maintained and wrinkle formation may be reduced.

The term “collagen” refers to a protein formed of a thousand or more amino acids and having a high content of hydroxyproline. Collagen fibers formed of three collagen molecules in a triple helix keep skin firm and elastic. In addition, collagen, as a main protein in various connective tissues in the body such as skin, blood vessels, bones, teeth, and muscles, is known to be involved in elasticity of skin.

The term “hyaluronic acid”, one of glycosaminoglycans, is a polymer of a polysaccharide chain in which glucuronic acid and N-acetyl glucosamine residues are repeatedly linked. Due to a property of binding to a large amount of water to form a gel, hyaluronic acid has high viscosity and elasticity. In addition, hyaluronic acid, as a main component of extracellular matrix, is known to be involved in moisture retention, intercellular spacing, storage and diffusion of cell growth factors and nutrients, as well as mitosis, differentiation, and migration of cells.

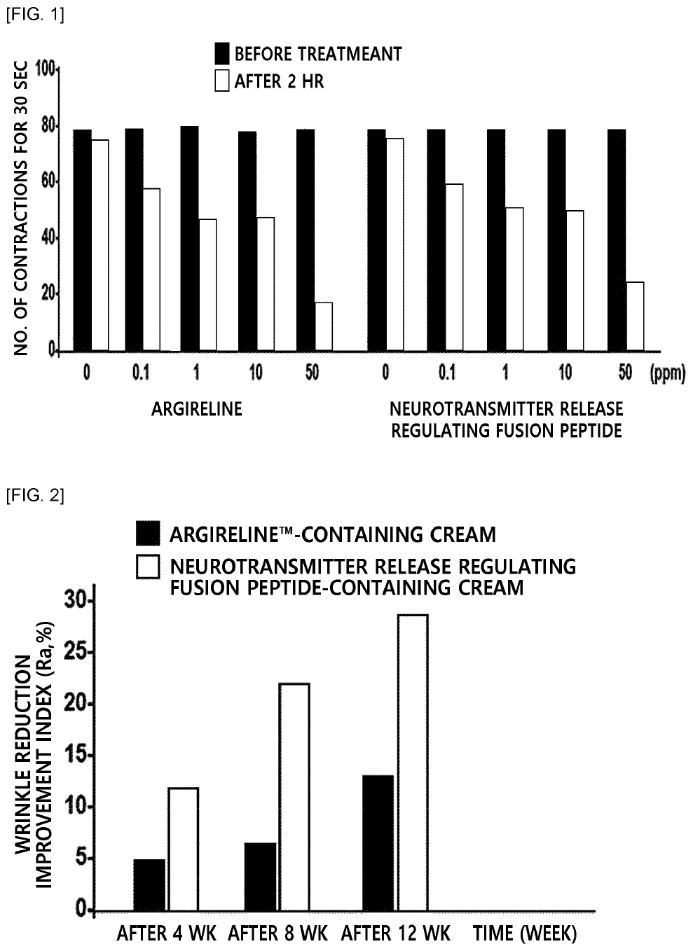

According to an exemplary embodiment, fusion peptides (SEQ ID NOS: 32, 33, 34, and botulinum toxin+SEQ ID NO: 1) were prepared by linking the neurotransmitter release regulating peptide, e.g., Argireline™ (Acetyl-Glu-Glu-Met-Gln-Arg-Arg, Acetyl-EEMQRR, SEQ ID NO: 2), Palmitoyl-hexapeptide-52 ([Pal]-Asp-Asp-Met-Gln-Arg-Arg, [Pal]DDMQRR SEQ ID NO: 3), Palmitoyl-heptapeptide-18 ([Pal]-Tyr-Pro-Trp-The-Gln-Arg-Phe ([Pal]YPWTQRF SEQ ID NO: 4)), and botulinum toxin, to the skin penetration enhancing peptide comprising the amino acid sequence of SEQ ID NO: 1. Upon comparison of effects of the prepared fusion proteins with those of conventional neurotransmitter release regulating peptides, it was confirmed that the prepared fusion proteins have excellent wrinkle reduction effects (FIG. 1).

According to an exemplary embodiment, effects of the fusion proteins according to the present invention on production of collagen and hyaluronic acid were tested to identify effects of the fusion proteins on skin elasticity enhancement and skin wrinkle reduction. As a result of culturing PDGFa and the fusion protein (T-PDGFa) in human dermal fibroblasts, it was confirmed that T-PDGFa also had the same level of effects on production of collagen and hyaluronic acid as those of PDGFa when compared with a control (Tables 4 and 5).

According to an exemplary embodiment, effects of the fusion protein according to the present invention on production of hyaluronic acid were tested to identify effects of the fusion protein on skin elasticity enhancement and skin wrinkle reduction. As a result of culturing VEGF and the fusion protein (T-VEGF) in human dermal fibroblasts, it was confirmed that T-VEGF also had the same level of hyaluronic acid production effect as VEGF when compared with a control (Table 9).

In addition, according to an exemplary embodiment, effects of the fusion protein according to the present invention on proliferation of umbilical vein endothelial cells were tested to identify effects of the fusion protein on skin elasticity enhancement and skin wrinkle reduction. As a result of culturing VEGF and the fusion protein (T-VEGF) in umbilical vein endothelial cells, it was confirmed that T-VEGF also had the same level of umbilical vein endothelial cell proliferation effect as VEGF when compared with a control (Table 9).

According to an exemplary embodiment, effects of the fusion protein according to the present invention on growth of keratinocytes were tested to identify effects of the fusion protein on skin elasticity enhancement and skin wrinkle reduction. As a result of culturing IGF-1 and the fusion protein (T-IGF-1) in skin keratinocytes, it was confirmed that T-IGF-1 also had the same level of keratinocyte growth effect as IGF-1 when compared with a control (Table 14).

According to an exemplary embodiment, effects of the fusion protein according to the present invention on growth of keratinocytes were tested to identify effects of the fusion protein on skin elasticity enhancement and skin wrinkle reduction. As a result of culturing KGF and the fusion protein (T-KGF) in skin keratinocytes, it was confirmed that T-KGF also had the same level of skin keratinocyte proliferation effect as KGF when compared with a control (Table 18).

According to an exemplary embodiment, effects of the fusion protein according to the present invention on proliferation of umbilical vein endothelial cells were tested to identify effects of the fusion protein on skin elasticity enhancement and skin wrinkle reduction. As a result of culturing Tβ4 and the fusion protein (T-Tβ4) in umbilical vein endothelial cells, it was confirmed that T-Tβ4 also had the same level of keratinocyte growth effect as Tβ4 when compared with a control (Table 22).

Based thereon, when the skin penetration enhancing peptide is linked to the physiologically active protein, e.g., the neurotransmitter release regulating peptide, PDGFa, VEGF, IGF-1, KGF, or Tβ4, it is confirmed that the effects of the physiologically active protein on skin wrinkle reduction and skin elasticity enhancement are maintained.

In addition, according to an embodiment of the present invention, fusion proteins (SEQ ID NOS: 32 to 34, 36, 39, 42, 45, and 48) were prepared by linking the skin penetration enhancing peptide comprising the amino acid sequence of SEQ ID NOS: 2 to 4, 35, 38, 41, 44, or 47 to the physiologically active protein (the neurotransmitter release regulating peptide, PDGFa, VEGF, IGF-1, KGF, and Tβ4). Upon comparison of effects of the prepared fusion proteins with those of conventional physiologically active proteins, it was confirmed that the prepared fusion proteins had excellent skin penetrability and skin retentivity and also excellent wrinkle reduction effects (Tables 1, 2, 7, 8, 12, 13, 16, 17, 20, 21, 24, and 25).

Thus, since the physiologically active protein is linked to the skin penetration enhancing peptide in the fusion proteins provided by the present invention, the effects of the physiologically active protein on enhancing regeneration of damage skin and hair may be maintained, as well as skin penetrability and skin retentivity may be improved. Therefore, the fusion proteins may be effectively used as active ingredients in cosmetic compositions, functional cosmetic products, and quasi-drug compositions.

The fusion protein of the present invention may be included in a cosmetic composition in an amount of 0.0001 wt % to 50 wt % based on the total weight of the cosmetic composition. When the amount of the fusion protein is less than 0.0001 wt % based on the total weight of the cosmetic composition, it is difficult to expect a substantial improvement in skin conditions. When the amount of the fusion protein is greater than 50 wt %, formulations may become unstable.

The cosmetic composition according to the present invention may be prepared into a formulation selected from a solution, ointment for external skin use, cream, foam, nourishing lotion, softening lotion, pack, skin softener, emulsion, makeup base, essence, soap, liquid cleanser, bath bomb, sun screen cream, sun oil, suspension, emulsion, paste, gel, lotion, powder, soap, surfactant-containing cleansing agent, oil, powder foundation, emulsion foundation, wax foundation, patch, and spray, without being limited thereto.

In addition, the cosmetic composition of the present invention may further include at least one cosmetically acceptable carrier mixed with general skin cosmetic formulations, such as oil, water, a surfactant, a moisturizing agent, a lower alcohol, a thickener, a chelating agent, a pigment, a preservative, and a fragrance, without being limited thereto.

The cosmetically acceptable carrier included in the cosmetic composition of the present invention may vary according to the formulation.

When the formulation of the present invention is an ointment, paste, cream, or gel, the carrier may be animal oil, vegetable oil, wax, paraffin, starch, tragacanth gum, cellulose derivative, polyethylene glycol, silicone, bentonite, silica, talc, zinc oxide, or any mixture thereof.

When the formulation of the present invention is a powder or spray, the carrier may be lactose, talc, silica, aluminum hydroxide, calcium silicate, polyamide powder, or any mixture thereof. Particularly, in the form of spray, the cosmetic composition may further include a propellent such as chlorofluorohydrocarbon, propane/butane, or dimethyl ether.

When the formulation of the present invention is a solution or emulsion, the carrier may be a solvent, a solubilizer, or an emulsifier, e.g., water, ethanol, isopropanol, ethyl carbonate, ethyl acetate, benzyl alcohol, benzyl benzoate, propylene glycol, and 1,3-butyl glycol oil, particularly, cotton seed oil, peanut oil, corn seed oil, olive oil, castor oil, and sesame oil, glycerol, aliphatic ester, polyethylene glycol, or fatty acid ester of sorbitan.

When the formulation of the present invention is a suspension, the carrier may be a liquid diluents such as water, ethanol, and propylene glycol, a suspending agents such as ethoxylated isostearyl alcohol, polyoxyethylene sorbitol ester, and polyoxyethylene sorbitan ester, microcrystalline cellulose, aluminum methahydroxide, bentonite, agar, or tragacanth gum.

When the formulation of the present invention is a soap, the carrier may be an alkali metal salt of fatty acid, hemiester salt of fatty acid, fatty acid protein hydrolysate, isethionate, lanolin derivative, aliphatic alcohol, vegetable oil, glycerol, or sugar.

According to an embodiment, a cream impregnated with the fusion protein prepared in which the skin penetration enhancing peptide is linked to the physiologically active protein was prepared. As a result of identifying the effects of the cream on improving skin conditions, it was confirmed that anti-wrinkle effects of the cream was more than twice the effects of a cream impregnated with the conventional physiologically active protein (Table 3 and FIG. 2).

Another aspect of the present invention provides a functional cosmetic product for improving skin conditions including the cosmetic composition as an active ingredient.

In addition, specifically, a functional cosmetic product including the cosmetic composition according to the present invention as an active ingredient. The cosmetic composition, skin wrinkle reduction, and skin elasticity enhancement are as described above.

As used herein, the term “functional cosmetic product (cosmedical or cosmeceutical)” refers to a cosmetic product that has special therapeutic effects of medical drugs, and thus shows special functionalities such as physiologically active effects, unlike general cosmetic products. The functional cosmetic product include products having effects on whitening or brightening skin, reducing wrinkles, tanning skin, and protecting skin from UV rays which are approved by the Ministry of Health and Welfare.

The functional cosmetic product of the present invention may further include an appropriate carrier used in preparation of general cosmetic products for skin. In this case, the carrier is not particularly limited and may specifically be oil, water, a surfactant, a moisturizing agent, a lower alcohol, a thickener, a chelating agent, a pigment, a preservative, and a fragrance, used alone or in combination.

The functional cosmetic product according to the present invention have skin wrinkle reduction or skin elasticity enhancement effects and may be prepared into a formulation such as a solution, emulsion, suspension, paste, cream, lotion, gel, powder, spray, surfactant-containing cleansing oil, soap, cleansing liquid, bath bomb, foundation, makeup base, essence, lotion, foam, pack, skin softener, sun screen cream, and sun oil, particularly, an ointment for external skin use, softening lotion, nourishing lotion, nourishing cream, massage cream, essence, emulsion, or oil gel, without being limited thereto. In this case, a carrier may be selectively used according to the formulation of cosmetic products.

For example, when the cosmetic product is in the form of ointment, paste, cream, or gel, the carrier may be wax, paraffin, starch, tragacanth gum, cellulose derivative, polyethylene glycol, silicone, bentonite, silica, talc, and zinc oxide which are used alone or in combination. When the cosmetic product is in the form of powder or spray, the carrier may be lactose, talc, silica, aluminum hydroxide, calcium silicate, polyamide powder, chlorofluorohydrocarbon, propane/butane, and dimethyl ether, which are used alone or in combination. When the cosmetic product is in the form of solution or emulsion, the carrier may be water, ethanol, isopropanol, ethyl carbonate, ethyl acetate, benzyl alcohol, benzyl benzoate, propylene glycol, 1,3-butyl glycol oil, cotton seed oil, peanut oil, corn seed oil, olive oil, castor oil, and sesame oil, glycerol, aliphatic ester, polyethylene glycol, and fatty acid ester of sorbitan which are used alone or in combination. When the cosmetic product is in the form of suspension, the carrier may be water, ethanol, propylene glycol, ethoxylated isostearyl alcohol, polyoxyethylene sorbitol ester, polyoxyethylene sorbitol ester sorbitan ester, microcrystalline cellulose, aluminum methahydroxide, bentonite, agar, and tragacanth gum which are used alone or in combination. When the cosmetic product is in the form of cosmetic soap, the carrier may be an alkali metal salt of fatty acid, hemiester salt of fatty acid, fatty acid protein hydrolysate, isethionate, lanolin derivative, aliphatic alcohol, vegetable oil, glycerol, and sugar which are used alone or in combination.

Specifically, an ointment for external skin use may further include, in addition to the fusion protein of the present invention, 50 wt % to 97 wt % of Vaseline and 0.1 wt % to 5 wt % of polyoxyethyleneoleyl-ether phosphate; the softening lotion may further include, in addition to the fusion protein of the present invention, 1 wt % to 10 wt % of a polyhydric alcohol such as propylene glycol or glycerin and 0.05 wt % to 2 wt % of a surfactant such as olyethyleneoleylether or polyoxyethylene hydrogenated castor oil; the nourishing lotion and nourishing cream may further include, in addition to the fusion protein of the present invention, 5 wt % to 20 wt % an oil such as squalane, Vaseline or octyldodecanol and 3 wt % to 15 wt % of a wax component such as cetanol, steelyl alcohol or beeswax; the essence may further include, in addition to the fusion protein of the present invention, 5 wt % to 30 wt % of a polyhydric alcohol such as glycerin or propylene glycol; the massage cream may further include, in addition to the fusion protein of the present invention, 30 wt % to 70 wt % of oil such as liquid paraffin, Vaseline or isononyl isononanoate; and the pack may be prepared as a peel-off pack further including, in addition to the fusion protein of the present invention, 5 wt % to 20 wt % of polyvinyl alcohol or as a wash-off pack further including 5 wt % to 30 wt % of a pigment such as kaolin, talc, zinc oxide, or titanium dioxide in addition to a general emulsion-type cosmetic formulation.

Another aspect of the present invention provides a cosmetic composition for preventing and treating alopecia including the fusion protein as an active ingredient.

As used herein, the term “preventing and treating alopecia” refers to preventing alopecia and stimulating hair growth. Alopecia, also known as hair loss, refers to a condition in which hairs are lost from part of the head or body where the hairs should be growing, and is caused by various factors such as genetic factors, hormone imbalance, mental stresses in daily life, air pollution, various habits such as eating of processed foods, and environmental influences.

According to an exemplary embodiment, dermal papilla cells treated with the fusion proteins of the present invention were cultured, and proliferated cells were quantitively analyzed using a Cell Counter Kit-8 (manufactured by Dojindo). Based on the analysis results, it was confirmed that the fusion protein in which the physiologically active protein is linked to the skin penetration enhancing peptide also exhibited the same level of effects on cell proliferation as that of the physiologically active protein when compared with a control (Tables 6, 11, 15, 19, and 23).

Thus, it can be seen that the effects of the physiologically active protein on prevention and treatment of alopecia are maintained although the physiologically active protein is linked to the skin penetration enhancing peptide.

The cosmetic composition may be prepared in a formulation of hair tonic, hair conditioner, hair essence, hair lotion, hair nourishing lotion, hair shampoo, hair rinse, hair treatment, hair cream, hair nourishing cream, hair moisturizing cream, hair massage cream, hair wax, hair aerosol, hair pack, hair nourishing pack, hair soap, hair cleansing foam, hair oil, hair drying preparation, hair preserving preparation, hair dye, hair waving preparation, hair gel, hair glaze, hair dressinger, hair lacquer, hair moisturizer, hair mousse, or hair spray, without being limited thereto.

Specifically, the composition according to the present invention may be used by using a method of directly applying or scatting onto the hair or scarp. Hairs to which the composition according to the present invention is applied may include hair roots and hair follicles of scarp, and all body hairs with roots and follicles such as scarp hair, eyelashes, eyebrows, mustaches, beards, armpit hair, and pubic hair.

Another aspect of the present invention provides a quasi-drug composition for improving skin conditions including the fusion protein as an active ingredient.

In addition, specifically, a quasi-drug composition for reducing skin wrinkle or enhancing skin elasticity including the fusion protein of the present invention as an active ingredient may be provided.

Another aspect of the present invention provides a quasi-drug composition for preventing and treating alopecia including the fusion protein as an active ingredient.

The terms fusion protein, skin wrinkle reduction, skin elasticity enhancement and prevention and treatment of alopecia are as described above.

The quasi-drug composition of the present invention may further include a pharmaceutically acceptable carrier, excipient, or diluent in addition to the above-described components. The pharmaceutically acceptable carrier, excipient or diluent is not limited as long as it does not adversely affect the effects of the present invention and may include a filler, an extender, a binder, a humectant, a disintegrant, a surfactant, a lubricant, a sweetener, a perfume, a preserving agent, and the like.

Examples of the pharmaceutically acceptable carrier, excipient or diluent according to the present invention may include lactose, dextrose, sucrose, sorbitol, mannitol, xylitol, maltitol, starch, gelatin, glycerine, Acacia rubber, alginate, calcium phosphate, calcium carbonate, calcium silicate, cellulose, methyl cellulose, amorphous cellulose, polyvinyl pyrrolidone, water, methyl hydroxy benzoate, propyl hydroxy benzoate, talc, magnesium stearate, mineral oils, propylene glycol, polyethylene glycol, vegetable oils, injectable esters, Witepsol, Macrogol, twin 61, cacao butter, and laurin butter.

In addition, when the composition including the fusion protein of the present invention as an active ingredient is used as a quasi-drug, the composition may further include one or more active agents having the same or similar function. For example, the composition may include components for protecting skin from damage, enhancing elasticity, reducing wrinkles, and moisturizing skin. When the components are added to the composition, safety to use on skin in combination, ease of formulation, and stability of active ingredients may be considered. The quasi-drug composition may further include one or more than two components selected from the group consisting of: skin lightening agents known in the art, e.g., a tyrosinase inhibitor, such as kojic acid and arbutin, hydroquinone, and vitamin C (L-Ascorbic acid); any known elasticity enhancing, wrinkle reducing, or moisturizing agents, e.g., retinoic acid, TGF, proteins derived from animal placenta, betulinic acid, and chlorella extracts; and derivatives thereof and various vegetable extracts. The additional component may be included in an amount of 0.0001 wt % to 5 wt % based on the total weight of the entire composition, and the amount range may be adjusted according to safety for use on skin, ease of application, and the like.

The quasi-drug composition of the present invention may be disinfecting detergent, shower foam, shower foam, ointment, wet tissue, coating agent, or the like, without being limited thereto. Formulating methods, dosages, methods of use, components, and the like may be appropriately selected from conventional techniques known in the art.

Also, the quasi-drug composition including the fusion protein according to the present invention as an active ingredient may be applied to skin of an individual for skin elasticity enhancement, skin wrinkle reduction, or prevention and treatment of alopecia. The individual may include mammals such as rats, livestock, and humans, without limitation.

### MODE OF DISCLOSURE

Hereinafter, the configuration and effects of the present invention will be described in more detail with reference to the following examples. However, these examples are for illustrative purposes only and are not intended to limit the scope of the present invention.

### Example 1: Selection of Skin Penetration Enhancing Peptide

In order to select a skin penetration enhancing peptide, a phage display method consisting of a combination of a phage library and elution test methods of a transdermal agent was performed.

First, 10⁹ phages derived from a Ph.D-12 phage library kit (New England Biolab) were added to 500 mL of a 1% BSA-containing TBS solution (50 mM Tris pH 7.5, 150 mM NaCl) to prepare a phage solution.

Subsequently, porcine skin (thickness: 0.7 mm, Medikinetics) was placed between upper and lower ends of Franz glass cell, (standard diameter: 9 mm, Receiver 5 mL, Permgear) and the phage solution was applied to the upper end, followed by reaction for 16 hours. Then, the phages that penetrated the porcine skin and reached a receiver located at the lower end were recovered and amplified.

The amplification was performed using E. coli ER2738 (New England Biolab) as a host cell. Specifically, 5 mL of the phage solution was added to an E. coli ER2738 stain shake-cultured in 25 mL of an LB medium and has cultured for 4 hours. Then, the culture solution was centrifuged at 8,000 G to obtain a supernatant containing phage fractions. The supernatant was reacted with 6 mL of a precipitation buffer (20%, PEG6000, 2.5 M NaCl) to precipitate the phages, and the reaction solution was centrifuged at 8,000 G to precipitate the phages. The precipitates were suspended in a TBS solution to obtain an amplified phage solution.

The above-described process of adding the phages to the porcine skin, collecting the phages that penetrated the skin and amplifying the collected phages was defined as Round 1. The phages amplified in Round 1 were subjected to Round 2 to select phages showing excellent skin penetrability in a competitive manner. A total of three rounds were performed.

In order to identify a sequence of a peptide contained in the phages finally obtained after Round 3, the TBS solution including the phages was added to the E. coli ER2738 stain and suspended, a TOP agar was added to the suspension and mixed, and the mixture was applied to an LB/X-gal/IPTG plate medium and solidified. The solidified medium was incubated for 16 hours, and then blue colonies were selected. Strains derived from the selected colonies were cultured for 6 hours, and DNA collected therefrom was analyzed to analyze a nucleotide sequence derived from the phages, thereby selecting a skin penetration enhancing peptide (SEQ ID NO: 1) exhibiting the property of penetrating the porcine skin.

### Example 2: Preparation of Fusion Protein in which Skin Penetration Enhancing Peptide is Linked to Physiologically Active Protein

### Example 2-1: Preparation of Fusion Protein Including Neurotransmitter Release Regulating Peptide and Skin Penetration Enhancing Peptide

A fusion protein having an amino acid sequence of SEQ ID NO: 32 in which the skin penetration enhancing peptide having the amino acid sequence of SEQ ID NO: 1 obtained in Example 1 is linked to a neurotransmitter release regulating peptide having an amino acid sequence of SEQ ID NO: 2, a fusion protein having an amino acid sequence of SEQ ID NO: 33 in which the skin penetration enhancing peptide having the amino acid sequence of SEQ ID NO: 1 is linked to a neurotransmitter release regulating peptide having an amino acid sequence of SEQ ID NO: 3, a fusion protein having an amino acid sequence of SEQ ID NO: 34 in which the skin penetration enhancing peptide having the amino acid sequence of SEQ ID NO: 1 is linked to a neurotransmitter release regulating peptide having an amino acid sequence of SEQ ID NO: 4, and a peptide having an amino acid sequence of botulinum toxin+SEQ ID NO: 1 in which the skin penetration enhancing peptide having the amino acid sequence of SEQ ID NO: 1 is linked to botulinum toxin, as a neurotransmitter release regulating peptide, were synthesized, followed by isolation and purification, to prepare neurotransmitter release regulating fusion proteins.

### Example 2-1-1: Synthesis of Neurotransmitter Release Regulating Fusion Peptide

The neurotransmitter release regulating fusion proteins were synthesized by solid-phase peptide synthesis using an Applied Biosystems Model 431A peptide synthesizer.

Specifically, 0.25 mmol of a parahydroxy methyloxymethyl polystyrene (HMP) resin was added to a standard reaction vessel (38 mL), and Fmoc-amino acid of the carboxy terminal of the peptide to be synthesized was added thereto to initiate synthesis. A cartridge containing 1 mmol of Fmoc-amino acid was arranged in a guideway in the sequence starting from the carboxy terminal amino acid to an end amino acid. In this case, metal openings of the cartridge were removed and empty cartridges without amino acids were laid on the first and last amino acids.

Before peptide synthesis, a parameter was edited according to a standard scale Fmoc coupling protocol developed by ABI Company, and peptide synthesis was conducted according to an autosynthesis menu (See ABI User's Manual. January, 1992). When using the standard scale Fmoc, deprotection was conducted for 21 minutes using 20% piperidine diluted with N-methylpyrrolidine (NMP), followed by washing with NMP for 9 minutes and coupling for 71 minutes. 1-hydroxy-benzotriazole (HOBT) was used for the coupling, and washing with NMP was conducted for an additional 7 minutes.

### Example 2-1-2: Separation and Purification of Fusion Protein

The neurotransmitter release regulating fusion proteins synthesized in Example 2-1-1 above were separated and purified according to the following process.

First, the fusion protein synthesized in Example 2-1-1 was separated from a solid support by using trifluoroacetic acid (TFA), as outlined in the ABI Company manual (Introduction to Cleavage Techniques, P6-19 1990). Specifically, a resin to which the fusion protein synthesized in Example 2-1-1 was added to a round-bottomed flask and cooled, and then 0.75 g of crystal phenol, 0.25 mL of 1,2-ethanedithiol (EDT), 0.5 mL of thioanisol, 0.5 mL of distilled water, and 10 mL of TFA were added to the flask and reacted at room temperature for 1 to 2 hours. After reaction, the resin and the reaction solution were filtered through a sintered glass funnel under low vacuum to separate the resin from the fusion protein solution. The flask and the glass funnel were washed with 5 mL to 10 mL dichloromethane (DCM) and the solution obtained from the washing was mixed with the fusion protein solution, and 50 mL or more of cool diethylether was added thereto to obtain a fusion protein precipitates. The precipitates were filtered through a funnel under low vacuum, and precipitates gathered on the funnel were dried, dissolved in 30% acetic acid, and lyophilized. The obtained fusion protein was purified by high performance liquid chromatography (HPLC). Here, a C18 analytical column (Pharmacia) was used, and buffer solution A including 10% acetonitrile and 0.05% TFA was used for equilibrium and buffer solution B including 80% acetonitrile and 0.05% TFA was used for elution of the fusion protein. As a result, a highly purified neurotransmitter release regulating fusion protein (SEQ ID NO: 32) was obtained with a synthesis yield of about 30±5%.

### Example 2-2: Preparation of Fusion Protein Including Platelet-derived Growth Factor Subunit a (PDGFa) and Skin Penetration Enhancing Peptide

A fusion protein T-PDGFa having an amino acid sequence of SEQ ID NO: 36 in which the C-terminal of the skin penetration enhancing peptide having the amino acid sequence of SEQ ID NO: 1 obtained in Example 1 is linked to the N-terminal of a platelet-derived growth factor subunit a (PDGFa) having an amino acid sequence of SEQ ID NO: 35 via a linker consisting of two amino acids (GG) was prepared.

Specifically, a polynucleotide encoding the amino acid sequence of SEQ ID NO: 36 was prepared by separately synthesizing a polynucleotide encoding the amino acid sequence of SEQ ID NO: 1 and a polynucleotide encoding the amino acid sequence of SEQ ID NO: 35, and then linking the polynucleotides via a nucleotide sequence encoding the two amino acids (GG). An expression vector was prepared by introducing the prepared polynucleotide into a pPIC expression vector.

The prepared expression vector was introduced into Pichia pastoris to obtain a transformant and the obtained transformant was cultured. Then, the culture solution was filtered to recover a fusion protein including the skin penetration enhancing peptide and VEGF. The recovered fusion protein was subjected to GPC column chromatography to prepare the final fusion protein T-PDGFa (SEQ ID NO: 36) including the skin penetration enhancing peptide and PDGFa.

### Example 2-3: Preparation of Fusion Protein Including Vascular Endothelial Growth Factor (VEGF) and Skin Penetration Enhancing Peptide

A fusion protein T-VEGF having an amino acid sequence of SEQ ID NO: 39 in which the C-terminal of the skin penetration enhancing peptide having the amino acid sequence of SEQ ID NO: 1 obtained in Example 1 is linked to the N-terminal of a vascular endothelial growth factor (VEGF) having an amino acid sequence of SEQ ID NO: 38 via a linker consisting of two amino acids (GG) was prepared.

Specifically, a polynucleotide encoding the amino acid sequence of SEQ ID NO: 39 was prepared by separately synthesizing a polynucleotide encoding the amino acid sequence of SEQ ID NO: 1 and a polynucleotide encoding the amino acid sequence of SEQ ID NO: 38, and then linking the polynucleotides via a nucleotide sequence encoding the two amino acids (GG). An expression vector was prepared by introducing the prepared polynucleotide into a pPIC expression vector.

The prepared expression vector was introduced into Pichia pastoris to obtain a transformant and the obtained transformant was cultured. Then, the culture solution was filtered to recover a fusion protein including the skin penetration enhancing peptide and VEGF. The recovered fusion protein was subjected to GPC column chromatography to prepare the final fusion protein T-VEGF (SEQ ID NO: 39) including the skin penetration enhancing peptide and VEGF.

### Example 2-4: Preparation of Fusion Protein Including Insulin-Like Growth Factor-I (IGFI) and Skin Penetration Enhancing Peptide

A fusion protein T-IGF-1 having an amino acid sequence of SEQ ID NO: 42 in which the C-terminal of the skin penetration enhancing peptide having the amino acid sequence of SEQ ID NO: 1 obtained in Example 1 is linked to the N-terminal of an insulin-like growth factor-1 (IGF-1) having an amino acid sequence of SEQ ID NO: 41 via a linker consisting of two amino acids (GG) was prepared.

Specifically, a polynucleotide encoding the amino acid sequence of SEQ ID NO: 42 was prepared by separately synthesizing a polynucleotide encoding the amino acid sequence of SEQ ID NO: 1 and a polynucleotide encoding the amino acid sequence of SEQ ID NO: 41, and then linking the polynucleotides via a nucleotide sequence encoding the two amino acids (GG). An expression vector was prepared by introducing the prepared polynucleotide into a pPIC expression vector.

The prepared expression vector was introduced into Pichia pastoris to obtain a transformant and the obtained transformant was cultured. Then, the culture solution was filtered to recover a fusion protein including the skin penetration enhancing peptide and VEGF. The recovered fusion protein was subjected to GPC column chromatography to prepare the final fusion protein T-IGF-1 (SEQ ID NO: 42) including the skin penetration enhancing peptide and IGF-1.

### Example 2-5: Preparation of Fusion Protein Including Keratinocyte Growth Factor (KGF) and Skin Penetration Enhancing Peptide

A fusion protein T-KGF having an amino acid sequence of SEQ ID NO: 45 in which the C-terminal of the skin penetration enhancing peptide having the amino acid sequence of SEQ ID NO: 1 obtained in Example 1 is linked to the N-terminal of a keratinocyte growth factor (KGF) having an amino acid sequence of SEQ ID NO: 44 via a linker consisting of two amino acids (GG) was prepared.

Specifically, a polynucleotide encoding the amino acid sequence of SEQ ID NO: 45 was prepared by separately synthesizing a polynucleotide encoding the amino acid sequence of SEQ ID NO: 1 and a polynucleotide encoding the amino acid sequence of SEQ ID NO: 44, and then linking the polynucleotides via a nucleotide sequence encoding the two amino acids (GG). An expression vector was prepared by introducing the prepared polynucleotide into a pPIC expression vector.

The prepared expression vector was introduced into Pichia pastoris to obtain a transformant and the obtained transformant was cultured. Then, the culture solution was filtered to recover a fusion protein including the skin penetration enhancing peptide and VEGF. The recovered fusion protein was subjected to GPC column chromatography to prepare the final fusion protein T-KGF (SEQ ID NO: 45) including the skin penetration enhancing peptide and KGF.

### Example 2-6: Preparation of Fusion Protein Including Thymosin Beta 4 (Tβ4) and Skin Penetration Enhancing Peptide

A fusion protein T-Tβ4 having an amino acid sequence of SEQ ID NO: 48 in which the C-terminal of the skin penetration enhancing peptide having the amino acid sequence of SEQ ID NO: 1 obtained in Example 1 is linked to the N-terminal of a thymosin beta 4 (Tβ4) having an amino acid sequence of SEQ ID NO: 47 via a linker consisting of two amino acids (GG) was prepared.

Specifically, a polynucleotide encoding the amino acid sequence of SEQ ID NO: 48 was prepared by separately synthesizing a polynucleotide encoding the amino acid sequence of SEQ ID NO: 1 and a polynucleotide encoding the amino acid sequence of SEQ ID NO: 47, and then linking the polynucleotides via a nucleotide sequence encoding the two amino acids (GG). An expression vector was prepared by introducing the prepared polynucleotide into a pPIC expression vector.

The prepared expression vector was introduced into Pichia pastoris to obtain a transformant and the obtained transformant was cultured. Then, the culture solution was filtered to recover a fusion protein including the skin penetration enhancing peptide and VEGF. The recovered fusion protein was subjected to GPC column chromatography to prepare the final fusion protein T-Tβ4 (SEQ ID NO: 48) including the skin penetration enhancing peptide and Tβ4.

### Example 3: Verification of Effect of Fusion Protein

### Example 3-1: Verification of Effect of Neurotransmitter Release Regulating Fusion Protein

In order to identify the use of the neurotransmitter release regulating fusion proteins prepared according to Example 2-1 for improvement of skin conditions, experiments to identify effects on inhibition of muscle contraction, skin penetrability, skin retentivity, and skin wrinkle reduction were conducted as follows.

3-1-1: Effect of Neurotransmitter Release Regulating Fusion Protein on Inhibition of Muscle Contraction

In order to identify the effects of the neurotransmitter release regulating fusion proteins prepared in Example 2-1 on inhibition of muscle contraction, first, C2C12 cells were cultured on a plate including a DMEM medium supplemented with a 10% (v/v) fetal bovine serum (FBS) and a 1% (v/v) antibiotic and additionally co-cultured with neuroblasts on the same plate. Then, the number of contractions of the C2C12 cells was measured for 30 second at the initial stage of cell contraction and all of the medium was removed, followed by washing three times with PBS. The cells were reacted for 2 hours in a medium supplemented with 50 ppm of the fusion peptide without the FBS. Then, the number of contractions of the C2C12 cells was measured for 30 seconds to identify the degree of inhibiting muscle contraction.

As a result, as shown in FIG. 1, it was confirmed that the number of contractions of the C2C12 cells was reduced not only in the control group using Argireline™ to which the skin penetration enhancing peptide was not bound but also in the neurotransmitter release regulating fusion peptide since the release of the neurotransmitter from nerve cells is inhibited.

3-1-2: Identification of Skin Penetrability of Neurotransmitter Release Regulating Fusion Protein

In order to identify skin penetrability of the neurotransmitter release regulating fusion proteins prepared in Example 2-1, Franz glass cell, (standard diameter: 9 mm, Receiver 5 mL, Permegear) were used.

Specifically, porcine skin (thickness: 0.7 mm, Medikinetics) was placed between upper and lower ends of the Franz glass cell, and TBS (50 mM Tris pH 7.5, 150 mM NaCl) including 1% BSA and 0.01% Tween 20 was prepared. Then, 500 μL of the TBS was applied to the upper end of the glass cell (Donor chamber), and 5 mL of the TBS was applied to the lower end of the glass cell, (Receiver chamber). Subsequently, 2 μg of the control peptide (Agirelline™, [Pal]DDMQRR, [Pal]YPWTQRF, and Botulinum toxoid) and 2 μg of each of the neurotransmitter release regulating fusion proteins were applied to the upper end of the glass cell, followed by reaction for 16 hours. A concentration of the control peptide or each of the neurotransmitter release regulating fusion proteins present in the lower end was quantitatively analyzed, and relative amounts of the neurotransmitter release regulating fusion peptides with respect to the amount of the control peptide were calculated, and the results are shown in Table 1 below.

TABLE 1
Skin penetrability of neurotransmitter
release regulating fusion proteins

| Treated peptide | Penetration enhancement (%) |
|---|---|
| Argireline ™ | 100 ± 14 |
| Fusion protein including Agirelline ™ | 360 ± 31 |
| (Acetyl-EEMQRR) | |
| Fusion protein including [Pal]DDMQRR | 420 ± 22 |
| Fusion protein including [Pal]YPWTQRF | 280 ± 15 |
| Fusion protein including Botulinum toxoid | 350 ± 22 |

As shown in Table 1 above, it was confirmed that the experimental groups treated with the neurotransmitter release regulating fusion protein had higher skin penetrability that that of the control group by about 2.8 to 4.2 times.

Therefore, it can be seen that the use of the neurotransmitter release regulating fusion proteins according to the present invention significantly increase skin penetrability of a neurotransmitter release regulating protein.

3-1-3: Identification of Skin Retentivity of Neurotransmitter Release Regulating Fusion Protein

In order to identify skin retentivity of the neurotransmitter release regulating fusion proteins prepared in Example 2-1, Franz glass cell, (standard diameter: 9 mm, Receiver 5 mL, Permegear) were used.

Specifically, porcine skin (thickness: 0.7 mm, Medikinetics) was placed between upper and lower ends of the Franz glass cell, and 1% BSA (Sigma) and 0.01% Tween 20 were dissolved respectively in 500 μL and 5 mL of the TBS (50 mM Tris pH 7.5, 150 mM NaCl). Then, 500 μL of the TBS was applied to the upper end of the glass cell, and 5 mL of the TBS was applied to the lower end of the glass cell. The control peptide (Agirelline™, [Pal]DDMQRR, [Pal]YPWTQRF, and Botulinum toxoid) and each of the neurotransmitter release regulating fusion protein prepared in Example 2 were added to the donor chamber of the Franz cell system containing the porcine skin, and the porcine skin tissue was disrupted and quantified by HPLC to measure the amounts of the control peptide and the neurotransmitter release regulating fusion proteins present in the porcine skin. The results are shown in Table 2 below.

TABLE 2
Skin retentivity of neurotransmitter
release regulating fusion proteins

| Treated peptide | Relative penetration (%) |
|---|---|
| Argireline ™ | 100 ± 14 |
| Fusion protein including Agirelline ™ | 9800 ± 1200 |
| (Acetyl-EEMQRR) | |
| Fusion protein including [Pal]DDMQRR | 7220 ± 422 |
| Fusion protein including [Pal]YPWTQRF | 6290 ± 1350 |
| Fusion protein including Botulinum toxoid | 8320 ± 242 |

As shown in Table 2 above, skin retention was increased by about 62 to 98 times when treated with the neurotransmitter release regulating fusion proteins compared to that of the control peptide.

Therefore, it can be seen that the use of the neurotransmitter release regulating fusion proteins according to the present invention increases skin retentivity of a neurotransmitter release regulating protein.

3-1-4: Identification of Effect of Neurotransmitter Release Regulating Fusion Protein on Skin Wrinkle Reduction

In order to identify the effects of the neurotransmitter release regulating fusion proteins prepared in Example 2-1 on skin wrinkle reduction, general-use oil-in-water emulsion-type creams were impregnated with Argireline™ and each of the neurotransmitter release regulating fusion proteins. The effects of the creams on skin wrinkle reduction were compared, and components included in the creams and amounts thereof are shown in Table 3 below.

TABLE 3

| | | Neurotransmitter |
|---|---|---|
| | | release regulating |
| | Argireline ™- | fusion peptide- |
| Component | containing cream | containing cream |
| mixture of C14-22 alcohol and | 1.5 | 1.5 |
| C12-20 alkyl glucoside (in a | | |
| weight ratio of 80:20) | | |
| mixture of glyceryl stearate and | 1.2 | 1.2 |
| PEG-100-stearate | | |
| (in a weight ratio of 50:50) | | |
| glyceryl stearate | 0.9 | 0.9 |
| cetearyl alcohol | 1.5 | 1.5 |
| polyglyceryl-3-methylglucose | 1.5 | 1.5 |
| distearate | | |
| hydrogenated polydecene | 4.5 | 4.5 |
| cyclohexasiloxane | 3.5 | 3.5 |
| carbomer | 0.2 | 0.2 |
| tromethamine | 0.2 | 0.2 |
| glycerin | 3 | 3 |
| DPG | 5 | 5 |
| 1,2-hexane diol | 2 | 2 |
| Argireline ™ | 0.1 | |
| neurotransmitter release | | 0.1 |
| regulating fusion peptide | | |
| purified water | Residual amount | Residual amount |
| | (To 100) | (To 100) |

Wrinkles around eyes were treated with the Argireline™-containing cream and the neurotransmitter release regulating fusion protein-containing cream every day for 12 weeks, and the degrees of reducing skin wrinkles were evaluated by silicone replica and wrinkle image analysis (N=21).

As a result, as shown in FIG. 2, the effects of the neurotransmitter release regulating fusion protein-containing cream on skin wrinkle reduction were more than twice higher than those of the Argireline™-containing cream, indicating the neurotransmitter release regulating fusion protein more effectively penetrates the skin than the neurotransmitter release regulating protein and has excellent skin wrinkle reduction effects.

This is because the neurotransmitter has a lower molecular weight than a conventional growth factor having a higher molecular weight. Since the fusion protein including the skin penetration enhancing peptide has a lower molecular weight, skin penetrability and skin retentivity thereof increases, thereby improving the skin wrinkle reduction effects.

### Example 3-2: Verification of Effect of Fusion Protein Including Platelet-Derived Growth Factor Subunit a

In order to identify the use of the T-PDGFa prepared in Example 2-2 for improvement of skin conditions, experiments to identify effects on skin elasticity enhancement, skin wrinkle reduction, prevention and treatment of alopecia, skin penetrability, and skin retentivity were conducted as follows.

### Example 3-2-1: Verification of Effect on Collagen Production

Effects of T-PDGFa synthesized in Example 2-2 on collagen production were verified in comparison with those of PDGFa.

Specifically, dermal fibroblasts were inoculated onto a 24-well plate and cultured for 24 hours to obtain cultures having a saturation degree of 70 to 80%. The cultures were washed with PBS and cultured in a serum-free DMEM medium including 10 ng/mL T-PDGFa or PDGFa for two days. A supernatant was obtained therefrom and the amount of collagen produced and released in the culture solution was quantitively analyzed using an ELISA kit (R&D Systems) (Table 4).

TABLE 4
Effect of fusion protein T-PDGFa on collagen production

| Treated protein | Production (%) |
|---|---|
| Control | 100 ± 3.7 |
| PDGFa | 264 ± 17 |
| T-PDGFa | 255 ± 15 |

As shown in Table 4, it was confirmed that T-PDGFa in which the skin penetration enhancing peptide was linked to PDGFa had the same level of collagen production effect as that of PDGFa.

Based thereon, it can be seen that the effects of PDGFa on skin wrinkle reduction and skin elasticity enhancement are maintained although the skin penetration enhancing peptide is linked to PDGFa.

### Example 3-2-2: Verification of Effect on Hyaluronic Acid (HA) Production

Effects of T-PDGFa synthesized in Example 2-2 on hyaluronic acid (HA) production were verified in comparison with those of PDGFa.

Specifically, dermal fibroblasts were inoculated onto a 24-well plate and cultured for 24 hours to obtain cultures having a saturation degree of 70 to 80%. The cultures were washed with PBS and cultured in a serum-free DMEM medium including 10 ng/mL T-PDGFa or PDGFa for two days. A supernatant was obtained therefrom and the amount of hyaluronic acid (HA) produced and released in the culture solution was quantitively analyzed using an ELISA kit (R&D Systems) (Table 5).

TABLE 5
Effect of fusion protein T-PDGFa on
hyaluronic acid (HA) production

| Treated protein | Production (%) |
|---|---|
| Control | 100 ± 4.9 |
| PDGFa | 897 ± 30 |
| T-PDGFa | 920 ± 29 |

As shown in Table 5, it was confirmed that T-PDGFa in which the skin penetration enhancing peptide was linked to PDGFa had the same level of hyaluronic acid (HA) producing effect as that of PDGFa.

Based thereon, it can be seen that the effects of PDGFa on skin wrinkle reduction and skin elasticity enhancement are maintained although the skin penetration enhancing peptide is linked to PDGFa.

### Example 3-2-3: Verification of Effect on Hair Follicle Stem Cell Proliferation

Effects of T-PDGFa synthesized in Example 2-2 on skin cell proliferation were verified in comparison with those of PDGFa.

Specifically, dermal papilla cells were inoculated onto a 96-well plate and cultured for 24 hours to obtain cultures at a density of 6,000 cells per well. The cultures were washed with PBS and cultured in a serum-free DMEM medium for one day. The cells were washed with PBS again and cultured in a serum-free DMEM medium supplemented with 1 ng/mL T-PDGFa or PDGFa for one day. The cultures were obtained therefrom and a difference between proliferated amounts of cells was quantitively analyzed using the Cell Counter Kit-8 (Dojindo). Here, dermal papilla cells cultured in a serum-free DMEM medium without T-PDGFa or PDGFa were used as a control (Table 6).

TABLE 6
Effect of fusion protein T-PDGFa on
hair follicle stem cell proliferation

| Treated protein | Proliferation (%) |
|---|---|
| control | 100 ± 11 |
| PDGFa | 1030 ± 19 |
| T-PDGFa | 1016 ± 7 |

As shown in Table 6, it was confirmed that T-PDGFa in which the skin penetration enhancing peptide was linked to PDGFa had the same level of cell proliferation effect as that of PDGFa.

Based thereon, it can be seen that the effects of PDGFa on prevention and treatment of alopecia are maintained although the skin penetration enhancing peptide is linked to PDGFa.

### Example 3-2-4: Verification of Skin Penetrability

Skin penetrability of T-PDGFa synthesized in Example 2-2 was verified in comparison with that of PDGFa.

Specifically, porcine skin (thickness: 0.7 mm, Medikinetics) was placed between upper and lower ends of Franz glass cell (standard diameter: 9 mm, Receiver 5 mL, Permegear), and TBS (50 mM Tris pH 7.5, 150 mM NaCl) including 1% BSA and 0.01% Tween 20 was prepared. Then, 500 μL of the TBS was applied to the upper end of the glass cell (Donor chamber), and 5 mL of the TBS was applied to the lower end of the glass cell, (Receiver chamber). Subsequently, 2 μg of PDGFa or T-PDGFa was applied to the upper end of the glass cell, followed by reaction for 16 hours. Then, a concentration of PDGFa or T-PDGFa was quantitatively analyzed using the ELISA kit (R&D Systems), and a relative amount of T-PDGFa to PDGFa was calculated as a penetrated amount (Table 7).

TABLE 7
Skin penetrability of fusion protein T-PDGFa

| Treated protein | Penetrated amount (%) |
|---|---|
| PDGFa | 100 ± 29 |
| T-PDGFa | 360 ± 45 |

As shown in Table 7, it was confirmed that T-PDGFa had about three times higher skin penetrability than PDGFa.

Based thereon, it can be seen that the use of fusion protein T-PDGFa according to the present invention significantly increases skin penetrability.

### Example 3-2-5: Verification of Skin Retentivity

Skin retentivity of fusion protein T-PDGFa synthesized in Example 2-2 were verified in comparison with that of PDGFa.

Specifically, the porcine skin remained after the experiment of Example 3-2-4 was recovered, frozen in liquid nitrogen, and disrupted. An amount of PDGFa or T-PDGFa contained therein was quantitatively analyzed using the ELISA kit (R&D Systems). In addition, a relative amount of T-PDGFa to PDGFa was calculated as a remaining amount (Table 8).

TABLE 8
Skin retentivity of fusion protein T-PDGFa

| Treated protein | Remaining amount (%) |
|---|---|
| PDGFa | 100 ± 20 |
| T-PDGFa | 9,500 ± 630 |

As shown in Table 8, it was confirmed that T-PDGFa had about 100 times higher skin retentivity than PDGFa.

Based thereon, it can be seen that the use of fusion protein T-PDGFa according to the present invention significantly increases skin retentivity.

### Example 3-3: Verification of Effect of Fusion Protein Including Vascular Endothelial Growth Factor (VEGF)

In order to identify the use of the T-VEGF prepared in Example 2-3 for improvement of skin conditions, experiments to identify effects on skin elasticity enhancement, skin wrinkle reduction, prevention and treatment of alopecia, skin penetrability, and skin retentivity were conducted.

### Example 3-3-1: Verification of Effect on Hyaluronic Acid (HA) Production

Effects of T-VEGF synthesized in Example 2-3 on hyaluronic acid (HA) production were verified in comparison with those of VEGF.

Specifically, dermal fibroblasts were inoculated onto a 24-well plate and cultured for 24 hours to obtain cultures having a saturation degree of 70 to 80%. The cultures were washed with PBS and cultured in a serum-free DMEM medium including 10 ng/mL T-VEGF or VEGF for two days. A supernatant was obtained therefrom and the amount of hyaluronic acid (HA) produced and released in the culture solution was quantitively analyzed using an ELISA kit (R&D Systems) (Table 9).

TABLE 9
Effect of fusion protein T-VEGF on
hyaluronic acid (HA) production

| Treated protein | Production (%) |
|---|---|
| Control | 100 ± 4.9 |
| VEGF | 220 ± 20 |
| T-VEGF | 230 ± 18 |

As shown in Table 9, it was confirmed that T-VEGF in which the skin penetration enhancing peptide was linked to VEGF had the same level of hyaluronic acid producing effect as that of VEGF.

Based thereon, it can be seen that the effects of VEGF on skin wrinkle reduction and skin elasticity enhancement are maintained although the skin penetration enhancing peptide is linked to VEGF.

### Example 3-3-2: Verification of Effect on Endothelial Cell Proliferation

Effects of T-VEGF synthesized in Example 2-3 on endothelial cell proliferation were verified in comparison with those of VEGF.

Specifically, human umbilical vein endothelial cells were inoculated onto a 96-well plate and cultured for 24 hours to obtain cultures at a density of 5,000 cells per well. The cultures were washed with PBS and cultured in a serum-free M199 medium for 16 hours. The cells were washed with PBS again and cultured in a serum-free M199 medium supplemented with 100 ng/mL T-VEGF or VEGF for one day. The cultures were obtained therefrom and a difference between proliferated amounts of cells was quantitively analyzed using the Cell Counter Kit-8 (Dojindo). Here, human umbilical vein endothelial cells cultured in a serum-free M199 medium without T-VEGF or VEGF were used as a control (Table 10).

TABLE 10
Effect of fusion protein T-VEGF on endothelial cell proliferation

| Treated protein | Proliferation (%) |
|---|---|
| Control | 100 ± 9.9 |
| VEGF | 170 ± 10 |
| T-VEGF | 185 ± 15 |

As shown in Table 6, it was confirmed that T-VEGF in which the skin penetration enhancing peptide was linked to VEGF had the same level of cell proliferation effect as that of VEGF.

Based thereon, it can be seen that the effects of VEGF on skin wrinkle reduction and skin elasticity enhancement are maintained although the skin penetration enhancing peptide is linked to VEGF.

### Example 3-3-3: Verification of Effect on Hair Follicle Stem Cell Proliferation

Effects of T-VEGF synthesized in Example 2-3 on skin cell proliferation were verified in comparison with those of VEGF.

Specifically, dermal papilla cells were inoculated onto a 96-well plate and cultured for 24 hours to obtain cultures at a density of 6,000 cells per well. The cultures were washed with PBS and cultured in a serum-free DMEM medium for one day. The cells were washed with PBS again and cultured in a serum-free DMEM medium supplemented with 1 ng/mL T-VEGF or VEGF for one day. The cultures were obtained therefrom and a difference between proliferated amounts of cells was quantitively analyzed using the Cell Counter Kit-8 (Dojindo). Here, dermal papilla cells cultured in a serum-free DMEM medium without T-VEGF or VEGF were used as a control (Table 11).

TABLE 11
Effect of fusion protein T-VEGF on hair
follicle stem cell proliferation

| Treated protein | Proliferation (%) |
|---|---|
| Control | 100 ± 4.9 |
| VEGF | 340 ± 50 |
| T-VEGF | 310 ± 20 |

As shown in Table 11, it was confirmed that T-VEGF in which the skin penetration enhancing peptide was linked to VEGF had the same level of cell proliferation effect as that of VEGF.

Based thereon, it can be seen that the effects of VEGF on prevention and treatment of alopecia are maintained although the skin penetration enhancing peptide is linked to VEGF.

### Example 3-3-4: Verification of Skin Penetrability

Skin penetrability of fusion protein T-VEGF synthesized in Example 2-3 was verified in comparison with that of VEGF.

Specifically, porcine skin (thickness: 0.7 mm, Medikinetics) was placed between upper and lower ends of Franz glass cell (standard diameter: 9 mm, Receiver 5 mL, Permegear), and TBS (50 mM Tris pH 7.5, 150 mM NaCl) including 1% BSA and 0.01% Tween 20 was prepared. Then, 500 μL of the TBS was applied to the upper end of the glass cell (Donor chamber), and 5 mL of the TBS was applied to the lower end of the glass cell, (Receiver chamber). Subsequently, 2 μg of VEGF or T-VEGF was applied to the upper end of the glass cell, followed by reaction for 16 hours. Then, a concentration of VEGF or T-VEGF was quantitatively analyzed using the ELISA kit (R&D Systems), and a relative amount of T-VEGF to VEGF was calculated as a penetrated amount (Table 12).

TABLE 12
Skin penetrability of fusion protein T-VEGF

| Treated protein | Penetrated amount (%) |
|---|---|
| VEGF | 100 ± 16 |
| T-VEGF | 295 ± 21 |

As shown in Table 12, it was confirmed that T-VEGF had about three times higher skin penetrability than VEGF.

Based thereon, it can be seen that the use of fusion protein T-VEGF according to the present invention significantly increases skin penetrability.

### Example 3-3-5: Verification of Skin Retentivity

Skin retentivity of fusion protein T-VEGF synthesized in Example 2-3 was verified in comparison with that of VEGF.

Specifically, the porcine skin remained after the experiment of Example 3-3-4 was recovered, frozen in liquid nitrogen, and disrupted. An amount of VEGF or T-VEGF contained therein was quantitatively analyzed using the ELISA kit (R&D Systems). In addition, a relative amount of T-VEGF to VEGF was calculated as a remaining amount (Table 13).

TABLE 13
Skin retentivity of fusion protein T-VEGF

| Treated protein | Remaining amount (%) |
|---|---|
| VEGF | 100 ± 24 |
| T-VEGF | 9,800 ± 790 |

As shown in Table 13, it was confirmed that T-VEGF had about 100 times higher skin retentivity than VEGF.

Based thereon, it can be seen that the use of fusion protein T-VEGF according to the present invention significantly increases skin retentivity.

### Example 3-4: Verification of Effect of Fusion Protein (T-IGF-1) Including Insulin-Like Growth Factor-1

In order to identify the use of the T-IGF-1 prepared in Example 2-4 for improvement of skin conditions, experiments to identify effects on skin elasticity enhancement, skin wrinkle reduction, prevention and treatment of alopecia, skin penetrability, and skin retentivity were conducted.

### Example 3-4-1: Verification of Effect on Skin Barrier Strengthening

Effects of T-IGF-1 synthesized in Example 2-4 on keratinocyte growth were verified in comparison with those of IGF-1.

Specifically, skin keratinocytes were cultured in a 96-well plate for one day to obtain cultures at a density of 6,000 cells per well. The cultures were washed with PBS and cultured in a serum-free DMEM medium for one day. The cells were washed with PBS again and cultured in a serum-free DMEM medium supplemented with 100 ng/mL T-IGF-1 or IGF-1 for one day. The cultures were obtained therefrom and a difference between proliferated amounts of cells was quantitively analyzed using the Cell Counter Kit-8 (Dojindo). Here, skin keratinocytes cultured in a serum-free DMEM medium without T-IGF-1 or IGF-1 were used as a control (Table 14).

TABLE 14
Effect of fusion protein T-IGF-1 on keratinocyte growth

| Treated protein | Production (%) |
|---|---|
| Control | 100 ± 12 |
| IGF-1 | 250 ± 20 |
| T-IGF-1 | 235 ± 15 |

As shown in Table 14, it was confirmed that T-IGF-1 in which the skin penetration enhancing peptide was linked to IGF-1 had the same level of skin wrinkle reduction effect as that of IGF-1.

### Example 3-4-2: Verification of Effect on Hair Follicle Stem Cell Proliferation

Effects of T-IGF-1 synthesized in Example 2-4 on skin cell proliferation were verified in comparison with those of IGF-1.

Specifically, dermal papilla cells were inoculated onto a 96-well plate and cultured for 24 hours to obtain cultures at a density of 6,000 cells per well. The cultures were washed with PBS and cultured in a serum-free DMEM medium for one day. The cells were washed with PBS again and cultured in a serum-free DMEM medium supplemented with 1 ng/mL T-IGF-1 or IGF-1 for one day. The cultures were obtained therefrom and a difference between proliferated amounts of cells was quantitively analyzed using the Cell Counter Kit-8 (Dojindo). Here, dermal papilla cells cultured in a serum-free DMEM medium without T-IGF-1 or IGF-1 were used as a control (Table 15).

TABLE 15
Effect of fusion protein T-IGF-1 on
hair follicle stem cell proliferation

| Treated protein | Proliferation (%) |
|---|---|
| control | 100 ± 15 |
| IGF-1 | 440 ± 45 |
| T-IGF-1 | 320 ± 40 |

As shown in Table 15, it was confirmed that T-IGF-1 in which the skin penetration enhancing peptide was linked to IGF-1 had the same level of cell proliferation effect as that of IGF-1.

Based thereon, it can be seen that the effects of IGF-1 on prevention and treatment of alopecia are maintained although the skin penetration enhancing peptide is linked to IGF-1.

### Example 3-4-3: Verification of Skin Penetrability

Skin penetrability of T-IGF-1 synthesized in Example 2-4 was verified in comparison with that of IGF-1.

Specifically, porcine skin (thickness: 0.7 mm, Medikinetics) was placed between upper and lower ends of Franz glass cell (standard diameter: 9 mm, Receiver 5 mL, Permegear), and TBS (50 mM Tris pH 7.5, 150 mM NaCl) including 1% BSA and 0.01% Tween 20 was prepared. Then, 500 μL of the TBS was applied to the upper end of the glass cell (Donor chamber), and 5 mL of the TBS was applied to the lower end of the glass cell, (Receiver chamber). Subsequently, 2 μg of IGF-1 or T-IGF-1 was applied to the upper end of the glass cell, followed by reaction for 16 hours. Then, a concentration of IGF-1 or T-IGF-1 was quantitatively analyzed using the ELISA kit (R&D Systems), and a relative amount of T-IGF-1 to IGF-1 was calculated as a penetrated amount (Table 16).

TABLE 16
Skin penetrability of fusion protein T-IGF-1

| Treated protein | Penetrated amount (%) |
|---|---|
| IGF-1 | 100 ± 14 |
| T-IGF-1 | 280 ± 18 |

As shown in Table 16, it was confirmed that T-IGF-1 had about three times higher skin penetrability than IGF-1.

Based thereon, it can be seen that the use of fusion protein T-IGF-1 according to the present invention significantly increases skin penetrability.

### Example 3-4-4: Verification of Skin Retentivity

Skin retentivity of fusion protein T-IGF-1 synthesized in Example 2-4 was verified in comparison with that of IGF-1.

Specifically, the porcine skin remained after the experiment of Example 3-4-4 was recovered, frozen in liquid nitrogen, and disrupted. An amount of IGF-1 or T-IGF-1 contained therein was quantitatively analyzed using the ELISA kit (R&D Systems). In addition, a relative amount of T-IGF-1 to IGF-1 was calculated as a remaining amount (Table 17).

TABLE 17
Skin retentivity of fusion protein T-IGF-1

| Treated protein | Remaining amount (%) |
|---|---|
| IGF-1 | 100 ± 28 |
| T-IGF-1 | 9,100 ± 650 |

As shown in Table 17, it was confirmed that T-IGF-1 had about 100 times higher skin retentivity than IGF-1.

Based thereon, it can be seen that the use of fusion protein T-IGF-1 according to the present invention significantly increases skin retentivity.

### Example 3-5: Verification of Effect of Fusion Protein (T-KGF) Including Keratinocyte Growth Factor

In order to identify the use of the T-KGF prepared in Example 2-5 for improvement of skin conditions, experiments to identify effects on skin elasticity enhancement, skin wrinkle reduction, prevention and treatment of alopecia, skin penetrability, and skin retentivity were conducted.

### Example 3-5-1: Verification of Effect on Skin Barrier Strengthening

Effects of T-KGF synthesized in Example 2-5 on keratinocyte growth were verified in comparison with those of KGF.

Specifically, skin keratinocytes were cultured in a 96-well plate for 24 hours to obtain cultures at a density of 6,000 cells per well.

The cultures were washed with PBS and cultured in a serum-free DMEM medium for one day. The cells were washed with PBS again and cultured in a serum-free DMEM medium supplemented with 100 ng/mL T-KGF or KGF for one day. The cultures were obtained therefrom and a difference between proliferated amounts of cells was quantitively analyzed using the Cell Counter Kit-8 (Dojindo). Here, skin keratinocytes cultured in a serum-free DMEM medium without T-KGF or KGF were used as a control (Table 18).

TABLE 18
Effect of fusion protein T-KGF on keratinocyte growth

| Treated protein | Production (%) |
|---|---|
| control | 100 ± 15 |
| KGF | 620 ± 25 |
| T-KGF | 590 ± 27 |

As shown in Table 18, it was confirmed that T-KGF in which the skin penetration enhancing peptide was linked to KGF had the same level of cell growth effect as that of KGF.

Based thereon, it can be seen that the effects of KGF on skin wrinkle reduction and skin elasticity enhancement are maintained although the skin penetration enhancing peptide is linked to KGF.

### Example 3-5-2: Verification of Effect on Hair Follicle Stem Cell Proliferation

Effects of T-KGF synthesized in Example 2-5 on skin cell proliferation were verified in comparison with those of KGF.

Specifically, dermal papilla cells were inoculated onto a 96-well plate and cultured for 24 hours to obtain cultures at a density of 6,000 cells per well. The cultures were washed with PBS and cultured in a serum-free DMEM medium for one day. The cells were washed with PBS again and cultured in a serum-free DMEM medium supplemented with 10 ng/mL T-KGF or KGF for one day. The cultures were obtained therefrom and a difference between proliferated amounts of cells was quantitively analyzed using the Cell Counter Kit-8 (Dojindo). Here, dermal papilla cells cultured in a serum-free DMEM medium without T-KGF or KGF were used as a control (Table 19).

TABLE 19
Effect of fusion protein T-KGF on hair
follicle stem cell proliferation

| Treated protein | Proliferation (%) |
|---|---|
| Control | 100 ± 18 |
| KGF | 360 ± 35 |
| T-KGF | 270 ± 15 |

As shown in Table 19, it was confirmed that T-KGF in which the skin penetration enhancing peptide was linked to KGF had the same level of cell proliferation effect as that of KGF.

Based thereon, it can be seen that the effects of KGF on prevention and treatment of alopecia are maintained although the skin penetration enhancing peptide is linked to KGF.

### Example 3-5-3: Verification of Skin Penetrability

Skin penetrability of T-KGF synthesized in Example 2-5 was verified in comparison with that of KGF.

Specifically, porcine skin (thickness: 0.7 mm, Medikinetics) was placed between upper and lower ends of Franz glass cell (standard diameter: 9 mm, Receiver 5 mL, Permegear), and TBS (50 mM Tris pH 7.5, 150 mM NaCl) including 1% BSA and 0.01% Tween 20 was prepared. Then, 500 μL of the TBS was applied to the upper end of the glass cell (Donor chamber), and 5 mL of the TBS was applied to the lower end of the glass cell, (Receiver chamber). Subsequently, 2 μg of KGF or T-KGF was applied to the upper end of the glass cell, followed by reaction for 16 hours. Then, a concentration of KGF or T-KGF was quantitatively analyzed using the ELISA kit (R&D Systems), and a relative amount of T-KGF to KGF was calculated as a penetrated amount (Table 20).

TABLE 20
Skin penetrability of fusion protein T-KGF

| Treated protein | Penetrated amount (%) |
|---|---|
| KGF | 100 ± 18 |
| T-KGF | 310 ± 12 |

As shown in Table 20, it was confirmed that T-KGF had about three times higher skin penetrability than KGF.

Based thereon, it can be seen that the use of fusion protein T-KGF according to the present invention significantly increases skin penetrability.

### Example 3-5-4: Verification of Skin Retentivity

Skin retentivity of fusion protein T-KGF synthesized in Example 2-5 was verified in comparison with that of KGF.

Specifically, the porcine skin remained after the experiment of Example 3-5-3 was recovered, frozen in liquid nitrogen, and disrupted. An amount of KGF or T-KGF contained therein was quantitatively analyzed using the ELISA kit (R&D Systems). In addition, a relative amount of T-KGF to KGF was calculated as a remaining amount (Table 21).

TABLE 21
Skin retentivity of fusion protein T-KGF

| Treated protein | Remaining amount (%) |
|---|---|
| KGF | 100 ± 17 |
| T-KGF | 9,700 ± 850 |

As shown in Table 21, it was confirmed that T-KGF had about 100 times higher skin retentivity than KGF.

Based thereon, it can be seen that the use of fusion protein T-KGF according to the present invention significantly increases skin retentivity.

### Example 3-6: Verification of Effect of Fusion Protein (T-Tβ4) Including Thymosin Beta 4

In order to identify the use of the T-Tβ4 prepared in Example 2-6 for improvement of skin conditions, experiments to identify effects on skin elasticity enhancement, skin wrinkle reduction, prevention and treatment of alopecia, skin penetrability, and skin retentivity were conducted.

### Example 3-6-1: Verification of Effect on Endothelial Cell Proliferation

Effects of T-Tβ4 synthesized in Example 2-6 on endothelial cell proliferation were verified in comparison with those of Tβ4.

Specifically, human umbilical vein endothelial cells were inoculated onto a 96-well plate and cultured for 24 hours to obtain cultures at a density of 5,000 cells per well. The cultures were washed with PBS and cultured in a serum-free M199 medium for 16 hours. The cells were washed with PBS again and cultured in a serum-free M199 medium supplemented with 100 ng/mL T-Tβ4 or Tβ4 for one day. The cultures were obtained therefrom and a difference between proliferated amounts of cells was quantitively analyzed using the Cell Counter Kit-8 (Dojindo). Here, human umbilical vein endothelial cells cultured in a serum-free M199 medium without T-Tβ4 or Tβ4 were used as a control (Table 22).

TABLE 22
Effect of fusion protein T-Tβ4 on endothelial cell proliferation

| Treated protein | Proliferation (%) |
|---|---|
| control | 100 ± 11 |
| Tβ4 | 180 ± 8 |
| T-Tβ4 | 165 ± 17 |

As shown in Table 22, it was confirmed that T-Tβ4 in which the skin penetration enhancing peptide was linked to Tβ4 had the same level of cell proliferation effect as that of Tβ4.

Based thereon, it can be seen that the effects of Tβ4 on skin wrinkle reduction and skin elasticity enhancement are maintained although the skin penetration enhancing peptide is linked to Tβ4.

### Example 3-6-2: Verification of Effect on Hair Follicle Stem Cell Proliferation

Effects of T-Tβ4 synthesized in Example 2-6 on skin cell proliferation were verified in comparison with those of Tβ4.

Specifically, dermal papilla cells were inoculated onto a 96-well plate and cultured for 24 hours to obtain cultures at a density of 6,000 cells per well. The cultures were washed with PBS and cultured in a serum-free DMEM medium for one day. The cells were washed with PBS again and cultured in a serum-free DMEM medium supplemented with 10 ng/mL T-Tβ4 or Tβ4 for one day. The cultures were obtained therefrom and a difference between proliferated amounts of cells was quantitively analyzed using the Cell Counter Kit-8 (Dojindo). Here, dermal papilla cells cultured in a serum-free DMEM medium without T-Tβ4 or Tβ4 were used as a control (Table 23).

TABLE 23
Effect of fusion protein T-Tβ4 on
hair follicle stem cell proliferation

| Treated protein | Proliferation (%) |
|---|---|
| Control | 100 ± 23 |
| Tβ4 | 260 ± 20 |
| T-Tβ4 | 220 ± 16 |

As shown in Table 23, it was confirmed that T-Tβ4 in which the skin penetration enhancing peptide was linked to Tβ4 had the same level of cell proliferation effect as that of Tβ4.

Based thereon, it can be seen that the effects of Tβ4 on prevention and treatment of alopecia are maintained although the skin penetration enhancing peptide is linked to Tβ4.

### Example 3-6-3: Verification of Skin Penetrability

Skin penetrability of fusion protein T-Tβ4 synthesized in Example 2-6 was verified in comparison with that of Tβ4.

Specifically, porcine skin (thickness: 0.7 mm, Medikinetics) was placed between upper and lower ends of Franz glass cell (standard diameter: 9 mm, Receiver 5 mL, Permegear), and TBS (50 mM Tris pH 7.5, 150 mM NaCl) including 1% BSA and 0.01% Tween 20 was prepared. Then, 500 μL of the TBS was applied to the upper end of the glass cell (Donor chamber), and 5 mL of the TBS was applied to the lower end of the glass cell, (Receiver chamber). Subsequently, 2 μg of Tβ4 or T-Tβ4 was applied to the upper end of the glass cell, followed by reaction for 16 hours. Then, a concentration of Tβ4 or T-Tβ4 was quantitatively analyzed using the ELISA kit (R&D Systems), and a relative amount of T-Tβ4 to Tβ4 was calculated as a penetrated amount (Table 24).

TABLE 24
Skin penetrability of fusion protein T-Tβ4

| Treated protein | penetrated amount (%) |
|---|---|
| Tβ4 | 100 ± 21 |
| T-Tβ4 | 310 ± 28 |

As shown in Table 24, it was confirmed that T-Tβ4 had about three times higher skin penetrability than Tβ4.

Based thereon, it can be seen that the use of fusion protein T-Tβ4 according to the present invention significantly increases skin penetrability.

### Example 3-6-4: Verification of Skin Retentivity

Skin retentivity of fusion protein T-Tβ4 synthesized in Example 2-6 was verified in comparison with that of Tβ4.

Specifically, the porcine skin remained after the experiment of Example 3-6-3 was recovered, frozen in liquid nitrogen, and disrupted. An amount of Tβ4 or T-Tβ4 contained therein was quantitatively analyzed using the ELISA kit (R&D Systems). In addition, a relative amount of T-Tβ4 to Tβ4 was calculated as a remaining amount (Table 25).

TABLE 25
Skin retentivity of fusion protein T-Tβ4

| Treated protein | Remaining amount (%) |
|---|---|
| Tβ4 | 100 ± 17 |
| T-Tβ4 | 9,600 ± 450 |

As shown in Table 25, it was confirmed that T-Tβ4 had about 100 times higher skin retentivity than Tβ4.

Based thereon, it can be seen that the use of fusion protein T-Tβ4 according to the present invention significantly increases skin retentivity.

The above description of the present invention is provided for the purpose of illustration, and it would be understood by those skilled in the art that various changes and modifications may be made without changing technical conception and essential features of the present invention. Thus, it is clear that the above-described embodiments are illustrative in all aspects and do not limit the present invention. The various embodiments disclosed herein are not intended to be limiting, with the true scope and spirit being indicated by the following claims. The present invention is to be limited only by the terms of the appended claims, along with the full scope of equivalents to which such claims are entitled.

1. A fusion protein comprising a skin penetration enhancing peptide comprising an amino acid sequence of SEQ ID NO: 1 and a physiologically active protein.

2. The fusion protein of claim 1, wherein the skin penetration enhancing peptide is bound to the N-terminal of the physiologically active protein.

3. The fusion protein of claim 1, wherein the physiologically active protein is selected from the group consisting of a neurotransmitter release regulating peptide, a platelet-derived growth factor subunit a (PDGFa), a vascular endothelial growth factor (VEGF), an insulin-like growth factor-1 (IGF-1), a keratinocyte growth factor (KGF), and a thymosin beta 4 (Tβ4).

4. The fusion protein of claim 3, wherein the neurotransmitter release regulating peptide comprises any one of amino acid sequences selected from the group consisting of SEQ ID NOS: 2 to 4, the platelet-derived growth factor subunit a (PDGFa) comprises an amino acid sequence of SEQ ID NO: 35, the vascular endothelial growth factor (VEGF) comprises an amino acid sequence of SEQ ID NO: 38, the insulin-like growth factor-1 (IGF-1) comprises an amino acid sequence of SEQ ID NO: 41, the keratinocyte growth factor (KGF) comprises an amino acid sequence of SEQ ID NO: 44, and the thymosin beta 4 (Tβ4) comprises an amino acid sequence of SEQ ID NO: 47.

5. The fusion protein of claim 4, wherein the neurotransmitter comprises at least one selected from the group consisting of dopamine, serotonin, histamine, acetylcholine, adrenaline, noradrenaline, gamma-aminobutyric acid (GABA), L-glutamic acid, and glycine.

6. The fusion protein of claim 1, wherein the fusion protein comprises any one of amino acid sequences selected from the group consisting of SEQ ID NOS: 32 to 34, 36, 39, 42, 45, and 48.

7. The fusion protein of claim 1, wherein the fusion protein has enhanced skin penetrability and skin retentivity of the physiologically active protein.

8. The fusion protein of claim 1, comprising a linker between the skin penetration enhancing peptide and the physiologically active protein.

9. A polynucleotide encoding the fusion protein according to claim 1.

10. A cosmetic composition for improving skin conditions, comprising the fusion protein according to claim 1 as an active ingredient.

11. The cosmetic composition of claim 10, wherein the improving skin conditions is skin wrinkle reduction or skin elasticity enhancement.

12. A functional cosmetic product for improving skin conditions, comprising the cosmetic composition of claim 10 as an active ingredient.

13. The functional cosmetic product of claim 12, wherein the improving skin conditions is skin wrinkle reduction or skin elasticity enhancement.

14. A cosmetic composition for preventing and treating alopecia, comprising the fusion protein according to claim 1 as an active ingredient.

15. A quasi-drug composition for improving skin conditions, comprising the fusion protein according to claim 1 as an active ingredient.

16. A quasi-drug composition for preventing and treating alopecia, comprising the fusion protein according to claim 1 as an active ingredient.


SEQUENCE LISTING (49 sequences)

SEQ ID NO: 1 - protein, 12 aa, Artificial Sequence
  Synthetic Peptide for skin permeation promotion
NGSLNTHLAPIL

SEQ ID NO: 2 - protein, 6 aa, Artificial Sequence
  Synthetic Acetyl-EEMQRR
  MOD_RES (1)..(1): Acetyl
EEMQRR

SEQ ID NO: 3 - protein, 6 aa, Artificial Sequence
  Synthetic [Pal]DDMQRR
  MOD_RES (1)..(1): Palmitoyl-hexapeptide-52 [Pal]
DDMQRR

SEQ ID NO: 4 - protein, 7 aa, Artificial Sequence
  Synthetic [Pal]YPWTQRF
  MOD_RES (1)..(1): Palmitoyl-heptapeptide-18 [Pal]
YPWTQRF

SEQ ID NO: 5 - protein, 21 aa, Artificial Sequence
  Synthetic peptide 5
MAEDADMRNELEEMQRRADQL

SEQ ID NO: 6 - protein, 23 aa, Artificial Sequence
  Synthetic peptide 6
ADESLESTRRMLQLVEESKDAGI

SEQ ID NO: 7 - protein, 13 aa, Artificial Sequence
  Synthetic peptide 7
ELEEMQRRADQLA

SEQ ID NO: 8 - protein, 12 aa, Artificial Sequence
  Synthetic peptide 8
ELEEMQRRADQL

SEQ ID NO: 9 - protein, 11 aa, Artificial Sequence
  Synthetic peptide 9
ELEEMQRRADQ

SEQ ID NO: 10 - protein, 10 aa, Artificial Sequence
  Synthetic peptide 10
ELEEMQRRAD

SEQ ID NO: 11 - protein, 9 aa, Artificial Sequence
  Synthetic peptide 11
ELEEMQRRA

SEQ ID NO: 12 - protein, 8 aa, Artificial Sequence
  Synthetic peptide 12
ELEEMQRR

SEQ ID NO: 13 - protein, 11 aa, Artificial Sequence
  Synthetic peptide 13
LEEMQRRADQL

SEQ ID NO: 14 - protein, 10 aa, Artificial Sequence
  Synthetic peptide 14
LEEMQRRADQ

SEQ ID NO: 15 - protein, 9 aa, Artificial Sequence
  Synthetic peptide 15
LEEMQRRAD

SEQ ID NO: 16 - protein, 8 aa, Artificial Sequence
  Synthetic peptide 16
LEEMQRRA

SEQ ID NO: 17 - protein, 7 aa, Artificial Sequence
  Synthetic peptide 17
LEEMQRR

SEQ ID NO: 18 - protein, 10 aa, Artificial Sequence
  Synthetic peptide 18
EEMQRRADQL

SEQ ID NO: 19 - protein, 9 aa, Artificial Sequence
  Synthetic peptide 19
EEMQRRADQ

SEQ ID NO: 20 - protein, 8 aa, Artificial Sequence
  Synthetic peptide 20
EEMQRRAD

SEQ ID NO: 21 - protein, 7 aa, Artificial Sequence
  Synthetic peptide 21
EEMQRRA

SEQ ID NO: 22 - protein, 6 aa, Artificial Sequence
  Synthetic peptide 22
EEMQRR

SEQ ID NO: 23 - protein, 13 aa, Artificial Sequence
  Synthetic peptide 23
LESTRRMLQLVEE

SEQ ID NO: 24 - protein, 12 aa, Artificial Sequence
  Synthetic peptide 24
NKDMKEAEKNLT

SEQ ID NO: 25 - protein, 6 aa, Artificial Sequence
  Synthetic peptide 25
KNLTDL

SEQ ID NO: 26 - protein, 26 aa, Artificial Sequence
  Synthetic peptide 26
IMEKADSNKTRIDEANQRATKMLGSG

SEQ ID NO: 27 - protein, 20 aa, Artificial Sequence
  Synthetic peptide 27
SNKTRIDEANQRATKMLGSG

SEQ ID NO: 28 - protein, 17 aa, Artificial Sequence
  Synthetic peptide 28
TRIDEANQRATKMLGSG

SEQ ID NO: 29 - protein, 14 aa, Artificial Sequence
  Synthetic peptide 29
DEANQRATKMLGSG

SEQ ID NO: 30 - protein, 11 aa, Artificial Sequence
  Synthetic peptide 30
NQRATKMLGSG

SEQ ID NO: 31 - protein, 10 aa, Artificial Sequence
  Synthetic peptide 31
QRATKMLGSG

SEQ ID NO: 32 - protein, 18 aa, Artificial Sequence
  Synthetic Fusion protein of Acetyl-EEMQRR
EEMQRRNGSLNTHLAPIL

SEQ ID NO: 33 - protein, 18 aa, Artificial Sequence
  Synthetic Fusion protein of [Pal]DDMQRR
DDMQRRNGSLNTHLAPIL

SEQ ID NO: 34 - protein, 19 aa, Artificial Sequence
  Synthetic Fusion protein of [Pal]YPWTQRF
YPWTQRFNGSLNTHLAPIL

SEQ ID NO: 35 - protein, 125 aa, Unknown
  Human PDGFa
SIEEAVPAVCKTRTVIYEIPRSQVDPTSANFLIWPPCVEVKRCTGCCNTSSVKCQPSRVHHRSVKVAKVEYVRKKPKLKEVQVRLEEHLECACATTSLNPDYREEDTGRPRESGKKRKRKRLKPT

SEQ ID NO: 36 - protein, 139 aa, Artificial Sequence
  Synthetic Fusion protein of T-PDGFa
NGSLNTHLAPILGGSIEEAVPAVCKTRTVIYEIPRSQVDPTSANFLIWPPCVEVKRCTGCCNTSSVKCQPSRVHHRSVKVAKVEYVRKKPKLKEVQVRLEEHLECACATTSLNPDYREEDTGRPRESGKKRKRKRLKPT

SEQ ID NO: 37 - DNA, 420 bp, Artificial Sequence
  Synthetic T-PDGFa
aacggctcccttaatacccatttggctccaatattgggaggttcaatcgaagaagctgtccctgcagtttgcaagacacgtactgtaatttatgagattcctcgaagtcaggttgatccgacctctgcaaattttttaatttggcccccatgcgttgaagtgaaaagatgtactggttgttgcaatacatccagcgtgaagtgtcaaccatcgcgtgttcatcacaggtctgttaaggtagccaaagttgaatacgtcagaaaaaagccaaaactgaaggaagtccaagttagacttgaagaacacttagagtgtgcttgtgccactacttcattgaaccctgattacagagaggaagacacgggaagaccaagagagtctggtaaaaaaagaaagaggaaaagactaaaacctacataa

SEQ ID NO: 38 - protein, 165 aa, Unknown
  Human VEGF
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 39 - protein, 179 aa, Artificial Sequence
  Synthetic Fusion protein of T-VEGF
NGSLNTHLAPILGGAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 40 - DNA, 550 bp, Artificial Sequence
  Synthetic T-VEGF
aatggaagtttgaacacccatctggcccctatacttggaggtgcaccaatggctgagggtggcggtcagaatcaccatgaagttgtcaaatttatggatgtttaccagagatcatattgccatcccatcgaaaccctcgtggatatatttcaggaataccctgatgaaattgagtatatcttcaagccctcttgtgtcccattaatgagatgtggtggatgttgtaatgatgaaggacttgaatgcgtaccaactgaggaatccaatattacaatgcaaatcatgaggattaagccacaccagggtcaacatattggggagatgtctttcctacaacacaacaagtgtgaatgcagaccaaaaaaggatagagctagacaagagaacccttgcggtccgtgctctgagagaagaaaacatttgtttgttcaagacccacaaacatgtaagtgctcatgtaaaaacactgactctcgatgtaaggctagacaattggaattgaacgagagaacttgtcgttgtgacaaacctaggcgttaatctagaataa

SEQ ID NO: 41 - protein, 70 aa, Unknown
  Human IGF1
GPETLCGAELVDALQFVCGDRGFYFNKPTGYGSSSRRAPQTGIVDECCFRSCDLRRLEMYCAPLKPAKSA

SEQ ID NO: 42 - protein, 84 aa, Artificial Sequence
  Synthetic Fusion protein of T-IGF1
NGSLNTHLAPILGGGPETLCGAELVDALQFVCGDRGFYFNKPTGYGSSSRRAPQTGIVDECCFRSCDLRRLEMYCAPLKPAKSA

SEQ ID NO: 43 - DNA, 252 bp, Artificial Sequence
  Synthetic T-IGF1
aacggttctttaaatacacatttggctccaattcttggcggaggtcctgagactctgtgtggagctgaactagttgatgccttacaattcgtttgtggtgatagaggattctattttaataaaccaaccgggtatggtagttcttctaggagagcaccccagactggtatcgttgatgaatgctgttttcgttcctgtgacttaagaagattggaaatgtactgtgcaccattgaagcctgctaaatcagct

SEQ ID NO: 44 - protein, 163 aa, Unknown
  Human KGF
CNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 45 - protein, 177 aa, Artificial Sequence
  Synthetic Fusion protein of T-KGF
NGSLNTHLAPILGGCNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 46 - DNA, 534 bp, Artificial Sequence
  Synthetic T-KGF
aacggctccttgaacacgcacttagctccaatcctgggtggatgtaatgacatgactcctgaacaaatggctactaacgttaattgttcttctcccgaaagacacactcgaagttacgattatatggagggcggtgatattagagtgagacgtcttttttgcaggacacaatggtacctccgtattgacaagagagggaaggtcaagggaacccaagaaatgaagaacaattacaacattatggagattaggactgttgccgttggaatcgttgctataaagggtgtggagtcagaattttatttggcaatgaataaggaaggtaagctgtatgccaagaaggaatgcaacgaggattgtaactttaaggagctaatattggagaaccattacaacacatatgcatctgccaagtggacccataatggaggtgaaatgttcgtagcattgaaccagaaaggaatccctgtcagaggtaagaagaccaagaaggaacagaagacagctcatttccttccaatggctattacttaa

SEQ ID NO: 47 - protein, 43 aa, Unknown
  Human T beta 4
SDKPDMAEIEKFDKSKLKKTETQEKNPLPSKETIEQEKQAGES

SEQ ID NO: 48 - protein, 57 aa, Artificial Sequence
  Synthetic Fusion protein of T-T beta 4
NGSLNTHLAPILGGSDKPDMAEIEKFDKSKLKKTETQEKNPLPSKETIEQEKQAGES

SEQ ID NO: 49 - DNA, 174 bp, Artificial Sequence
  Synthetic T-T beta 4
aatggttcattgaacacacatttggccccaattcttggaggttccgacaagcctgatatggcagagattgaaaaattcgacaaatctaagttgaaaaaaactgaaacccaagagaagaaccccctgccatctaaggaaactatcgagcaagagaagcaggctggtgaaagttaa